(19) United States
(12) Patent Application Publication
(10) Pub. No.: US 2025/0026698 A1
(43) Pub. Date: Jan. 23, 2025

(54) ACTIVATION OF CATALYST SYSTEMS FOR THE PRODUCTION OF LIQUEFIED PETROLEUM GAS (LPG) HYDROCARBONS FROM SYNTHESIS GAS

(71) Applicant: GTI Energy, Des Plaines, IL (US)

(72) Inventors: Patrick LITTLEWOOD, Chicago, IL (US); Terry MARKER, Park Ridge, IL (US); Michael BRADFORD, Des Plaines, IL (US)

(21) Appl. No.: 18/224,397

(22) Filed: Jul. 20, 2023

(51) Int. Cl. C07C 1/04; B01J 21/04; B01J 23/80; B01J 29/70; B01J 37/08

(52) U.S. Cl. CPC C07C 1/043; B01J 21/04; B01J 23/80; B01J 29/7007; B01J 37/08; C07C 2521/04; C07C 2523/06; C07C 2523/72; C07C 2523/80; C07C 2529/70

(57) ABSTRACT

Processes are disclosed for the production of liquefied petroleum gas (LPG) hydrocarbons, utilizing both alcohol (e.g., methanol) synthesis and in situ dehydration of the alcohol to hydrocarbons, and particularly propane and/or butane. Operational adjustments and/or variations, involving activation of LPG synthesis catalyst systems at relatively high temperatures, followed by initial operation at relatively low temperatures, surprisingly lead to improvements in the stability of such catalyst systems. These improvements are often in conjunction with other benefits, for example greater selectivity and/or per-pass yield, with such improvements and benefits resulting from the ability to operate at reduced temperatures. Problems typically encountered in the art due to catalyst instability, such as the need for frequent replacement and/or regeneration, are thereby potentially reduced or even eliminated.

### FIELD OF THE INVENTION

Aspects of the invention relate to the activation of catalyst systems, for use in the conversion of synthesis gas comprising H₂ and CO to products comprising propane and/or butane, for example those having a composition approximating that of liquefied petroleum gas (LPG).

### DESCRIPTION OF RELATED ART

The ongoing search for alternatives to crude oil, as a conventional source of carbon for hydrocarbon products, is increasingly driven by a number of factors. These include diminishing petroleum reserves, higher anticipated energy demands, and heightened concerns over greenhouse gas (GHG) emissions from sources of non-renewable carbon. Hydrocarbon products of greatest industrial significance and interest, in terms of having their carbon content replaced with non-petroleum derived carbon, include transportation and heating fuels as well as precursors for specialty chemicals. The particular hydrocarbons propane and/or butane are present in many of these products, a common example of which is liquefied petroleum gas (LPG).

A key commercial process for converting methane, biomass, coal, or other carbonaceous feedstocks into fuels involves a first conversion step to produce synthesis gas (syngas), followed by a second, downstream Fischer-Tropsch (FT) conversion step. With respect to the first conversion step, known processes for the production of syngas include partial oxidation reforming and autothermal reforming (ATR), based on the exothermic oxidation of methane with oxygen. Steam methane reforming (SMR), in contrast, uses steam as the oxidizing agent, such that the thermodynamics are significantly different, not only because the production of steam itself can require an energy investment, but also because reactions involving methane and water are endothermic. More recently, it has also been proposed to use carbon dioxide as the oxidizing agent for methane, such that the desired syngas is formed by the reaction of carbon in its most oxidized form (CO₂) with carbon in its most reduced form (CH₄). This reaction has been termed the “dry reforming” of methane, and because it is highly endothermic, thermodynamics for the dry reforming of methane are less favorable compared to ATR or even SMR. Gasification and pyrolysis have also been in extensive use for converting both renewable and non-renewable sources of carbon (e.g., biomass and coal) into syngas. A technology for processing diverse types of solid feedstocks including biomass, municipal solid waste, and plastics, which yields syngas in combination with deoxygenated hydrocarbon products suitable for use as gasoline and/or diesel fuel, is known as hydropyrolysis and is described in U.S. Pat. Nos. 8,492,600 and 10,619,105, as well as other patents assigned to Gas Technology Institute (Des Plaines, IL).

With respect to the second step involving FT conversion, synthesis gas containing a mixture of hydrogen and carbon monoxide (CO) is subjected to successive cleavage of C—O bonds and formation of C—C bonds with the incorporation of hydrogen. This mechanism provides for the formation of hydrocarbons, and particularly straight-chain alkanes with a distribution of molecular weights that can be controlled to some extent by varying the FT reaction conditions (temperature and feed H₂:CO ratio) and catalyst properties. Such properties include pore size and other characteristics of the support material. The choice of catalyst can impact FT product yields in other respects. For example, iron-based FT catalysts tend to produce more oxygenates, whereas ruthenium as the active metal tends to produce exclusively paraffins. The reaction pathways of FT synthesis follow a statistical kinetic model, which leads to hydrocarbons having an Anderson-Schultz-Flory distribution of their carbon numbers. In the case of targeting the C₃ and C₄ hydrocarbons, i.e., propane and butane, this generally involves operating in a low conversion regime with a significant co-production of methane and ethane. Higher conversions, on the other hand, generate C₅⁺ hydrocarbons that are liquid at room temperature. Additional potential routes for the production of LPG hydrocarbons from syngas are described by K. Asami et al. (STUDIES IN SURFACE SCIENCE AND CATALYSIS 147 (2004) 427-432); Q. Zhang et al. (FUEL PROCESSING TECHNOLOGY 85 (2004) 1139-1150); and Q. Ge et al. (JOURNAL OF MOLECULAR CATALYSIS A: CHEMICAL 278 (2007) 215-219.

In terms of known pathways offering the potential for producing LPG hydrocarbons from synthesis gas, which is desirably derived from renewable methane (e.g., present in biogas) or biomass, improvements are needed in a number of areas. These include reaction product selectivity and yield, in combination with catalyst stability, all of which parameters significantly impact commercial viability. Overall, the state of the art would benefit from technologies for the efficient conversion of industrially available sources of synthesis gas, whether obtained as a standalone feed or from an upstream processing stage (e.g., a reforming stage) of an integrated process, to products comprising propane and/or butane. Industrially relevant examples of such products are those having a composition approximating that of liquefied petroleum gas (LPG). With respect to the practical impact of such technologies, a current objective of a number of countries around the world is to reduce deforestation and the generation of pollution, both of which result from the burning of wood for heating and cooking. However, because of the remoteness of many locations and the associated, long transportation routes, petroleum-derived LPG is priced at a premium and therefore not considered, from a cost standpoint, an acceptable alternative to wood. Accordingly, a number of significant advantages could be gained by efficiently obtaining LPG hydrocarbons from renewable resources that provide synthesis gas. These advantages include freedom from the need to import petroleum-derived LPG, a reduction in GHG emissions, improvement in air quality, and the potential stimulation of local economies, particularly in poorer regions.

### SUMMARY OF THE INVENTION

Aspects of the invention are associated with the discovery of liquefied petroleum gas (LPG) synthesis catalyst systems, and the manner in which such systems are utilized, to provide both alcohol (e.g., methanol) synthesis and in situ dehydration of the alcohol (e.g., methanol) to hydrocarbons, and particularly the LPG hydrocarbons propane and/or butane. Particular aspects relate to operational adjustments and/or variations that surprisingly lead to improvements in the stability of such catalyst systems, often in conjunction with other benefits, such as greater selectivity and/or per-pass yield, with such improvements and benefits resulting from the ability to operate at reduced temperatures. Problems typically encountered in the art due to catalyst instability, such as the need for frequent replacement and/or regeneration, are thereby potentially reduced or even eliminated.

For example, known processes for the conversion of methanol to olefinic hydrocarbons (methanol-to-olefins, or MTO, processes) using solid acid catalysts require continuous catalyst regeneration (CCR) to address the problem of rapid catalyst coking. Due to stability improvements associated with LPG synthesis catalyst systems, coupled with the manner in which they are implemented, as described herein, in representative embodiments these catalyst systems may be utilized in a fixed bed configuration, or otherwise in an alternative bed configuration (e.g., as a fluidized bed), but without continuous catalyst regeneration. In any event, those skilled in the art will appreciate that even modest increases in catalyst stability, selectivity, and/or per-pass yield will generally translate to very significant economic benefits on a commercial scale. In addition to relaxed requirements for catalyst regeneration, such benefits may be attributed, for example, to a decreased formation of undesired byproducts, including catalyst coke precursors, and/or reduced recycle gas requirements. Relevant to all of these desired improvements, the ability to lower operating temperature is often instrumental in avoiding non-selective side reactions that lead to coking and that are detrimental to catalyst stability.

Accordingly, in particular embodiments, synthesis gas is converted to an LPG product comprising a mixture of propane and butanes with a high selectivity, meaning that byproducts (e.g., methane, ethane, and/or C₅⁺ hydrocarbons) are generated in relatively small quantities. Such selectivity improvements and corresponding catalyst stability improvements may result from the ability to effectively utilize a lower initial operation temperature, while still achieving favorable conversion levels. Without being bound by theory, the conversion pathway proceeds through a methanol and/or dimethyl ether (DME) intermediate, potentially utilizing a “hydrocarbon pool” mechanism associated with methanol-to-hydrocarbons and/or DME-to-hydrocarbons reactions. According to a specific, representative mechanism, a zeolite (e.g., zeolite beta, ZSM-5, or SSZ-13) or zeotype (e.g., SAPO-34) component of a heterogeneous catalyst system, in combination with a variety of methylated aromatic species within the catalyst pore volume, can produce hydrocarbons via a range of reactions, including any one or more of dehydration, methylation, dealkylation, alkylation/beta scission, protonation/deprotonation, hydride and methyl shift, isomerization, cracking, and hydrogenation. It is postulated that, first, synthesis gas is converted to an oxygenate (e.g., an alcohol such as methanol and/or an ether such as DME) over a methanol synthesis catalyst or constituent, whereas a second catalyst or constituent (e.g., intimately mixed with the first) catalyzes the conversion of that oxygenate to light alkanes.

Merely for purposes of explanation and without limiting the invention to any particular mechanism, it is believed that, in order for the second, oxygenate-to-light alkane reaction to occur, the initial formation of the hydrocarbon pool within the pore volume of the second catalyst or constituent (e.g., an acidic component such as a zeolite or zeotype) may require relatively high temperatures (e.g., within a range of 310° C.-350° C.). In contrast, at lower temperatures (e.g., within a range of 250° C.-310° C.) substantially any methanol formed from the activity of the first, methanol synthesis catalyst or constituent, is converted by the activity of the second catalyst to DME, with only a minimal amount of hydrocarbon production. Accordingly, temperatures significantly above 300° C., such as in the range of 400-450° C., would be an expected reaction condition for obtaining the desired LPG products with acceptable yields.

Against this expectation, aspects of the invention relate to the surprising discovery that the synthesis gas-to-LPG hydrocarbon pathway can be “activated” or “primed” to allow for favorable performance at lower temperatures. This may be achieved by first operating at moderate reaction temperatures (e.g., in the range from 300° C.-400° C.) for a relatively short period, sufficient for forming the hydrocarbon pool. After such activation, reaction temperature may then be lowered. In the absence of such activation, operating at the given post-activation starting temperature (which would simply correspond in this case to the starting temperature, such as about 290° C.) would result in a yield of predominantly DME from the combined effects of the first and second catalysts or constituents as described above. In contrast, by first generating the hydrocarbon pool at the higher activation temperature, and then cooling the LPG synthesis reactor and contained catalyst system to the initial operation temperature, the selectivity to, and yield of, LPG hydrocarbons remains surprisingly high, even at temperatures that would otherwise fail to cause appreciable hydrocarbon production.

This ability to utilize a lower initial operation temperature, following an effective activation (e.g., to establish the hydrocarbon pool), while nonetheless maintaining favorable conversion, selectivity, and yield characteristics, has significant implications for the economic viability of converting syngas to LPG hydrocarbons. Directionally, lower operating temperatures are conducive to catalyst stability, whereas higher operating temperatures are conducive to catalyst deactivation. For example, typical methanol synthesis catalysts, such as those based generally on copper-zinc oxide-alumina systems, deactivate rapidly by metal particle sintering at higher temperatures. Also, catalysts in general, and zeolites more particularly, deactivate at least to some extent by coking, with coke-forming reactions from byproducts proceeding at higher rates as the temperature increases. Aside from plugging active sites as a catalyst surface contaminant, coke can take the form of solid species that accumulate within the hydrocarbon pool which, as noted above, is believed to be instrumental in methanol-to-hydrocarbons chemistry. At higher temperatures, these solid species are generated and grow in size more rapidly. In contrast, by establishing the hydrocarbon pool in an activation step, followed by a temperature reduction to the initial operation temperature, this type of coking mechanism may be significantly hampered or avoided altogether. Regardless of the particular coking mechanism(s) that is/are implicated, in view of any such mechanism(s) being exacerbated to varying extents at elevated temperatures, the ability to operate at lower temperatures, at least initially or as a “start of run” condition, has the significant beneficial effect of lowering the catalyst deactivation rate, as a result of reduced coking. Improved catalyst stability, in turn, directly favors process economics due to a number of potential effects, including improved selectivity/yield over a lengthened operating period, lower heating demands, reduced downtime, and/or decreased reactor construction material (i.e., capital) costs.

Moreover, the methanol synthesis reaction is thermodynamically limited, with higher temperatures corresponding to lower concentrations of methanol in the vapor phase (i.e., due to lower per-pass conversion and yield). The ability to lower operating temperature, therefore, relaxes equilibrium constraints associated with methanol synthesis, allowing a further route to process improvement. Additionally, thermodynamic limitations with respect to methanol synthesis can translate to reduced productivity over the course of normal temperature control of a catalytic process, whereby the reaction temperature is increased gradually to maintain overall performance of the catalyst as it deactivates. A lower initial operation temperature can help counteract this problem, by offering a greater period of on-stream operation under conditions that are more favorable from a thermodynamic standpoint. As a further consideration, in view of the sequential nature of coupled, methanol synthesis and LPG synthesis reactions, with the latter proceeding more readily at higher temperatures, a given set of conditions may cause these reactions to become unbalanced or otherwise restricted. The initial generation of the hydrocarbon pool, for example in an activation step whereby the catalyst system is contacted with an activating gas, allows temperature to be utilized to a greater extent for process control, such as in maintaining a desired degree of productivity through continual or intermittent raising of temperature throughout the usable life of the catalyst system.

By first generating the hydrocarbon pool via an activation step, and thereafter decreasing temperature to initiate or continue LPG synthesis, process control and benefits associated with operational stability, as described herein, can be achieved. Importantly, such benefits are otherwise not attainable by simply starting the methanol synthesis/LPG synthesis reactions at the decreased temperature, without activation or formation of the hydrocarbon pool. That is, activation of the catalyst system leads to surprising and unexpected advantages. According to certain embodiments, LPG hydrocarbons (as opposed to methanol and/or DME) may be produced in significant yields, via methanol and/or DME acting as reaction intermediate(s), at lower temperatures or at least lower initial operation temperatures (e.g., less than 300° C.) than achieved without activation or otherwise according to conventional operating modes.

Overall aspects of the invention therefore relate to the discovery of certain process steps that allow the conversion of synthesis gas to LPG hydrocarbons at a relatively low initial operation temperature. Utilizing these steps, the rate of deactivation of catalyst systems, for example one or more individual catalyst types used in such systems, may be thereby substantially decreased. According to some embodiments, whereas some deficits in reaction rate may be realized as a matter of kinetics from operating at initially lower temperatures, such deficits may be significantly outweighed by the longer-term effects associated with a decreased catalyst deactivation rate and associated, longer operating life over the range of gradually increasing temperatures that can be used to manage productivity.

Representative LPG synthesis catalyst systems have activities for both alcohol (e.g., methanol) and/or ether (e.g., DME) synthesis, as well as dehydration. These catalyst systems may comprise two catalyst types (e.g., in a macroscopically uniform mixture of particles) or otherwise a bi-functional catalyst (e.g., having a macroscopically uniform, particle-to-particle composition) comprising two types of functional constituents. In the case of two catalyst types (e.g., separate compositions, each being in the form of separate particles), these may include both an alcohol (e.g., methanol) synthesis catalyst and a dehydration catalyst, the latter of which may alternatively be referred to as an alcohol to LPG hydrocarbon conversion (ATLPG) catalyst, such as in the case of a methanol to LPG hydrocarbon conversion (MTLPG) catalyst. The separate catalyst types may be present in a given catalyst system (e.g., contained in an LPG synthesis reactor) in the form of a mixture, in the form of individual beds of one type or another (e.g., one or more beds of an alcohol synthesis catalyst alone, and/or one or more beds of a dehydration catalyst alone), or a combination thereof (e.g., one or more beds of a mixture, and/or one or more beds of alcohol synthesis catalyst alone and/or one or more beds of dehydration catalyst alone). In one embodiment, a bed of an alcohol (e.g., methanol) synthesis catalyst may precede (e.g., be positioned upstream of) a bed of a dehydration catalyst. In the case of a bi-functional catalyst, the functional constituents may include both an alcohol (e.g., methanol) synthesis-functional constituent and a dehydration-functional constituent, the latter of which may alternatively be referred to as an alcohol to LPG hydrocarbon conversion-(ATLPG-) functional constituent, such as in the case of a methanol to LPG hydrocarbon conversion-(MTLPG-) functional constituent.

Embodiments of the invention are directed to a process for producing an LPG product comprising propane and/or butane (and preferably both), the process comprising contacting a synthesis gas comprising H₂ and CO with an LPG synthesis catalyst system as described herein, and particularly such catalyst system comprising either separate catalysts or a bi-functional catalyst. In representative processes, the LPG synthesis catalyst systems described herein may be used to provide novel pathways for the production of liquefied petroleum gas (LPG) products comprising propane and/or butane, and in certain cases renewable LPG products, i.e., in which some or all (e.g., at least about 70%) of their carbon content (whether expressed on a wt-% or mole-% basis) is renewable carbon that is not derived from petroleum. Advantageously, whether or not the carbon content is renewable carbon, at least a portion (e.g., at least about 20%, at least about 30%, or at least about 40%) of the total carbon content of representative LPG products described herein may be derived from CO₂, for example being present as a component of a methane-containing gaseous feed mixture (e.g., biogas) that is subjected to upstream reforming or otherwise being present as a component of a gasification or pyrolysis effluent. In the case of a non-renewable carbon content that is derived from CO₂, such CO₂ may be obtained, for example, as a fossil fuel combustion product or a fossil fuel reforming product. In either case, it can be appreciated that CO₂ used to provide at least a portion of the total carbon content is beneficially utilized as LPG, rather than being directly released into the atmosphere. Within the environment of an LPG synthesis reactor containing the catalyst system, CO₂ may be present in an equilibrium or non-equilibrium amount, together with H₂, CO, and H₂O as other reactants/products of the reversible water-gas shift (WGS) reaction.

Particular embodiments of the invention are directed to a process for producing an LPG product comprising propane and/or butane. Representative processes comprise (a) an activation step, followed by (b) an operating step and may more specifically comprise (a) contacting an activating gas (e.g., comprising H₂ and CO, or otherwise comprising a hydrocarbon or oxygenated hydrocarbon) with an LPG synthesis catalyst system at an activation temperature or above (e.g., a threshold temperature for establishing a hydrocarbon pool in pores of a dehydration catalyst or dehydration-functional constituent), and thereafter (b) contacting a synthesis gas comprising H₂ and CO with the LPG synthesis catalyst system at an initial operation temperature that is less than the activation temperature. Other representative processes comprise contacting a synthesis gas comprising H₂ and CO with an LPG synthesis catalyst system at an LPG synthesis catalyst system temperature that is a pre-activation temperature, sufficient to provide a pre-activation methanol and/or dimethyl ether (DME) yield (e.g., a pre-activation methanol and/or DME yield of at least about 20%); (a) increasing the LPG synthesis catalyst system temperature to an activation temperature or above (e.g., above the pre-activation temperature), sufficient to provide an activation LPG hydrocarbon yield of at least about 30%; and (b) decreasing the LPG synthesis catalyst system temperature to an initial operation temperature, sufficient to maintain an initial operation LPG hydrocarbon yield of at least about 30%. In this case, the flow of synthesis gas may be maintained during steps (a) and (b), with the synthesis gas used in these steps having the same composition as, or possibly a different composition from, the synthesis gas used for contacting at the pre-activation temperature. As noted above, the LPG synthesis catalyst system used in any representative process described herein may comprise: (i) an alcohol synthesis catalyst, and (ii) a dehydration catalyst. Alternatively, the LPG synthesis catalyst system may comprise (i) an alcohol synthesis-functional constituent, and (ii) a dehydration-functional constituent.

These and other embodiments, aspects, and advantages relating to the present invention are apparent from the following Detailed Description.

### BRIEF DESCRIPTION OF THE DRAWINGS

A more complete understanding of the exemplary embodiments of the present invention and the advantages thereof may be acquired by referring to the following description in consideration of the accompanying figures.

FIG. 1 illustrates the effect of activation of an LPG synthesis catalyst system, in terms of the subsequent, favorable LPG hydrocarbon yield at temperatures below that used for activation.

FIG. 2 illustrates the effect of activation in the LPG synthesis reaction to obtain the results shown in FIG. 1, but in terms of the subsequent, favorable CO conversion and LPG hydrocarbon selectivity at temperatures below that used for activation.

FIG. 3 illustrates activation of an LPG synthesis catalyst system for LPG hydrocarbon production, utilizing a different temperature profile over time, compared to that used in obtaining the results shown in FIGS. 1 and 2.

### DETAILED DESCRIPTION

The expressions “wt-%” and “mol-%,” are used herein to designate weight percentages and molar percentages, respectively. The expressions “wt-ppm” and “mol-ppm” designate weight and molar parts per million, respectively. For ideal gases, “mol-%” and “mol-ppm” are equal to percentages by volume and parts per million by volume, respectively. In some cases, a percentage, “%,” is given with respect to values that are the same, whether expressed as a weight percentage or a molar percentage. For example, the percentage of the carbon content of the LPG product that is renewable carbon, has the same value, whether expressed as a weight percentage or a molar percentage.

The term “substantially,” as used herein, refers to an extent of at least 95%. For example, the phrase “substantially all” may be replaced by “at least 95%.”

A “synthesis gas comprising H₂ and CO,” or more simply “synthesis gas,” as described herein, may be representative of a portion of, or the entirety of, the material that is fed or input, e.g., that is input in one feed stream, or in two or more separate or combined feed streams, to an LPG synthesis reactor, used to carry out the conversion of at least a portion of the H₂ and CO to propane and/or butane that is contained in an LPG product. The synthesis gas comprising H₂ and CO may be, or may comprise, in particular embodiments, a synthesis gas intermediate, or portion thereof, which is produced in an upstream reaction stage, such as a stage for carrying out reforming to generate the H₂ and CO. Whether or not obtained from a synthesis gas intermediate, at least a portion of the H₂ and CO in the synthesis gas may be converted by contact with an LPG synthesis catalyst system as described herein, to propane and/or butane that is contained in the LPG product. This conversion may proceed through a mechanism whereby an alcohol (e.g., methanol) produced from H₂ and CO (according to an alcohol synthesis reaction) is dehydrated to LPG hydrocarbons and water. In view of the hydrogen requirement for alcohol (e.g., methanol) synthesis and dehydration, the synthesis gas may have an H₂:CO molar ratio of at least about 2.0, such as from about 2.0 to about 2.5. Such molar ratios may be obtained, optionally following an adjustment (e.g., increase) occurring upstream of the conversion of the synthesis gas (e.g., upstream of an LPG synthesis reactor).

Alternatively to, or in combination with, alcohol synthesis, the conversion of synthesis gas to LPG hydrocarbons may proceed through a mechanism whereby an ether (e.g., DME) is produced. For example, in the case of a combination, methanol or other alcohol produced initially may be dehydrated to DME or other ether, which is then further dehydrated to LPG hydrocarbons. Accordingly, the terms “alcohol synthesis catalyst” and “alcohol synthesis-functional constituent” should be understood to refer to catalysts and functional constituents that may catalyze, or at least lead to (mechanistically), the formation of ethers (e.g., DME), alternatively to, or in combination with, the formation of alcohols (e.g., methanol).

Any source of synthesis gas comprising H₂ and CO may be used as a feed to an LPG synthesis reactor, in representative LPG synthesis processes, including a synthesis gas that is produced at least partly by reforming. The synthesis gas may comprise H₂ and CO in any suitable amounts (concentrations), such as in combined amount of greater than about 25 mol-% (e.g., from about 25 mol-% to 100 mol-%), greater than about 50 mol-% (e.g., from about 50 mol-% to about 99 mol-%), or greater than about 75 mol-% (e.g., from about 75 mol-% to about 99 mol-%). With respect to any such combined amounts (concentrations), the H₂:CO molar ratio of the synthesis gas may be from about 1.0 to about 7.0, such as from about 4.0 to about 6.5, in the case of relatively high ratios. Otherwise, in the case of relatively low ratios, the H₂:CO molar ratio of the synthesis gas intermediate may be from about 1.0 to about 3.0, such as from about 1.8 to about 2.4. The LPG product, comprising propane (C₃H₈) and/or butane (C₄H₁₀), may be obtained using catalyst systems as described herein for catalyzing reactions of methanol synthesis and dehydration, as follows:

14H₂+7CO→7CH₃OH (methanol synthesis), and

7CH₃OH+2H₂→C₃H₈+C₄H₁₀+7H₂O (dehydration).

According to the above reactions, the LPG hydrocarbons propane and butane may be produced from synthesis gas through a methanol intermediate. As noted above, LPG hydrocarbons may also be produced from synthesis gas through a DME intermediate, such as in the case of H₂ and CO reacting to form DME (CH₃OCH₃) and water, followed by dehydration of DME to LPG hydrocarbons. Otherwise, LPG hydrocarbons may be produced from synthesis gas through both a methanol intermediate and a DME intermediate, such as in the case of H₂ and CO reacting to form methanol, followed by dehydration of methanol to DME, and further dehydration of DME to LPG hydrocarbons.

Alternatively, or in combination, CO₂ present in the synthesis gas may likewise advantageously be reacted in an initial methanol synthesis, or in an initial DME synthesis, according to a second pathway. For example, in the case of producing the same number of moles of CH₃OH shown in the first reaction above, CO₂, rather than CO, may be consumed according to:

21H₂+7CO₂→7CH₃OH+7H₂O (methanol synthesis).

In view of any of these proposed routes to LPG hydrocarbons, the synthesis gas may have an H₂:CO molar ratio of at least about 1.0 (e.g., from about 1.0 to about 3.5 or from about 1.5 to about 3.0), or more preferably at least about 2.0 (e.g., from about 2.0 to about 4.0, from about 2.0 to about 3.0, or from about 2.0 to about 2.5). In some cases, excess H₂ (i.e., H₂ in excess of the stoichiometric amount needed to react with CO and/or CO₂ to form a methanol intermediate according to the reactions above, or otherwise a DME intermediate) may be desired to improve stability of a given LPG synthesis catalyst system.

More generally, the LPG product comprising propane and/or butane may be produced through synthesis of a methanol intermediate or higher alcohol intermediate, obtained from the reaction of H₂ with CO or CO₂, according to the following generalized reactions:

2n H₂+n CO→CₙH₂ₙ₊₁OH+(n−1)H₂O and/or

(3n+b)H₂+(n+b)CO₂→CₙH₂ₙ₋₁OH+(2n+b−1)H₂O+b CO (alcohol synthesis), and

(7/n)CₙH₂ₙ₊₁OH+2H₂→C₃H₈+C₄H₁₀+(7/n)H₂O (dehydration).

According to the above reactions, the LPG hydrocarbons propane and butane may be produced from synthesis gas, more generally through an alcohol intermediate. As noted above, LPG hydrocarbons may also be produced from synthesis gas more generally through an ether intermediate, such as in the case of H₂ and CO reacting to form an ether (e.g., CₙH₂ₙ₋₁O CₙH₂ₙ₊₁) and water, followed by dehydration of the ether to LPG hydrocarbons. Otherwise, LPG hydrocarbons may be produced from synthesis gas through both an alcohol intermediate and an ether intermediate, such as in the case of H₂ and CO reacting to form an alcohol, followed by dehydration of the alcohol to the ether, and further dehydration of the ether to LPG hydrocarbons. Whereas the above reactions are illustrative of possible pathways for obtaining LPG hydrocarbons from synthesis gas, these are to be construed as exemplary reactions, without limitation of embodiments of the invention, unless expressly included as elements of such embodiments. Those skilled in the art having knowledge of the present disclosure will appreciate that these and other possible pathways, including other reaction stoichiometries, may be used to explain observed results of obtaining LPG hydrocarbons from synthesis gas.

Independently of, or in combination with, the representative amounts (concentrations) of H₂ and CO above and/or representative H₂:CO molar ratios above, the synthesis gas may further comprise CO₂, for example in an amount of at least about 5 mol-% (e.g., from about 5 mol-% to about 50 mol-%), at least about 10 mol-% (e.g., from about 10 mol-% to about 35 mol-%), or at least about 15 mol-% (e.g., from about 15 mol-% to about 30 mol-%). In such cases, the balance of the synthesis gas may be, or may substantially be, H₂ and CO in combination, for example in an H₂:CO molar ratio as described herein.

In the processing of a synthesis gas comprising H₂ and CO, catalyst systems as described herein can provide important advantages in terms of stability, leading to process economics favorable for commercialization. In particular, the use of an activation step, at a relatively high activation temperature, may be used to “activate” or “prime” an LPG synthesis catalyst system described herein, such as by establishing a hydrocarbon pool that is necessary for the continued production of LPG hydrocarbons, even following a temperature reduction to a relatively low initial operation temperature. Representative processes for producing an LPG product comprising propane and/or butane may therefore comprise (a) contacting an activating gas with an LPG synthesis catalyst system at an activation temperature or above, and thereafter (b) contacting a synthesis gas comprising H₂ and CO with the LPG synthesis catalyst system at an initial operation temperature that is less than the activation temperature. According to some embodiments, the LPG synthesis catalyst system may comprise (i) an alcohol synthesis catalyst, such as a methanol synthesis catalyst and (ii) a dehydration catalyst. Alternatively, the LPG synthesis catalyst system may comprise a bi-functional catalyst, having as constituents (i) an alcohol synthesis-functional constituent, such as a methanol synthesis-functional constituent, and (ii) a dehydration-functional constituent. According to such representative processes, the step (a) may be considered an “activation step” and the step (b) may be considered an “operating step.” The contacting of the activating gas in step (a) therefore occurs at an activation temperature that exceeds the initial operation temperature in step (b). Such activation temperature in step (a) may lead to advantages as described above, for example in terms of allowing for favorable performance (e.g., selectivity to and/or yield of LPG hydrocarbons) at the initial operation temperature in step (b), which performance may otherwise not be attainable in the absence of the activation step (a). Without being bound by theory, this performance advantage associated with the activation step (a) may result from establishing a hydrocarbon pool as described above.

A representative activating gas comprises one of more of H₂, CO, and CO₂, such as in the case of being a mixture comprising, consisting essentially of, or consisting of, any one, two, or three of these components (e.g., comprising or consisting of a mixture of H₂ and CO). For example, the activating gas may comprise, or consist of, H₂ and CO. In this regard, a suitable activating gas may correspond to any “synthesis gas comprising H₂ and CO” as described above. Therefore, whereas, in some embodiments, both the activating gas in step (a) and the synthesis gas in step (b) may be characterized as a “synthesis gas comprising H₂ and CO” as described above, the activating gas and the synthesis gas may have different compositions, such as in the case of having one or more different components, or possibly having the same components but in different concentrations, or relative amounts (e.g., different H₂:CO molar ratios). According to specific embodiments, for convenience, the activating gas and the synthesis gas may have the same composition, such that, for example, steps (a) and (b) may be distinguishable on the basis of the different activation temperature and initial operation temperature alone.

Whereas activating gases comprising one of more of H₂, CO, and CO₂ (e.g., comprising H₂ and CO only) may be effective for activating or priming the LPG synthesis catalyst system according to step (a), such as by establishing the hydrocarbon pool, in some embodiments representative activating gases may generate one or more of H₂, CO, and CO₂ (e.g., in situ, upon contacting of the activating gas at the activation temperature with the LPG synthesis catalyst system). The generation of one or more of H₂, CO, and CO₂ may occur, for example, by reaction of the activating gas over the LPG synthesis catalyst system and/or by decomposition of the activating gas (or components of the activating gas such as oxygenated hydrocarbons). Particular activating gases generate H₂ and CO. Types of activating gases include hydrocarbons, such as C₁-C₅ hydrocarbons, including straight-chain C₁-C₅ paraffinic and olefinic hydrocarbons, as well as branched C₁-C₅ paraffinic and olefinic hydrocarbons (e.g., methane, ethane, ethylene, propane, propylene, butanes, butenes, pentanes, and pentenes). Other hydrocarbons include C₆-C₁₂ aromatic hydrocarbons, having one or more benzene rings. Exemplary activating gases may therefore comprise benzene, methyl-substituted benzenes (e.g., toluene and xylenes), naphthalene, and methyl-substituted naphthalenes. In addition to, or as an alternative to, one or more hydrocarbons, the activating gas may comprise one or more oxygenated hydrocarbons, with particular examples being alcohols such as methanol and ethanol; aldehydes such as formaldehyde and acetaldehyde; ketones, such as acetone and methyl ethyl ketone; and carboxylic acids such as formic acid and acetic acid. In addition to, or as an alternative to, one or more hydrocarbons having a structure comprising a benzene ring, suitable activating gases may comprise one or more oxygenated hydrocarbons having a structure comprising a benzene ring, with phenol and cresols being particular examples. Particular hydrocarbons and oxygenated hydrocarbons, as components of activating gases, may be liquid at room temperature but nonetheless gases under conditions used in an activation step (a), including temperature, total pressure, and partial pressure(s) of these components.

The activation temperature, at or above which the activating gas is contacted with the LPG synthesis catalyst system for its activation, may be considered a threshold temperature, at or above which this system becomes activated or primed for producing LPG hydrocarbons, meaning that a lower initial operation temperature can achieve a given level of performance (e.g., selectivity to and/or yield of LPG hydrocarbons), compared to a reference initial operation temperature that would be required in the absence of the activation step. Alternatively, at a given initial operation temperature, a higher level of performance (e.g., selectivity to and/or yield of LPG hydrocarbons) may be achieved following activation, compared to a reference level of performance in the absence of the activation step.

According to particular embodiments, the activation temperature may be at least about 295° C. (e.g., from about 295° C. to about 400° C., from about 295° C. to about 375° C., from about 295° C. to about 350° C., or from about 295° C. to about 325° C.); the activation temperature may be at least about 300° C. (e.g., from about 300° C. to about 400° C., from about 300° C. to about 375° C., from about 300° C. to about 350° C., or from about 300° C. to about 325° C.); the activation temperature may be at least about 305° C. (e.g., from about 305° C. to about 400° C., from about 305° C. to about 375° C., from about 305° C. to about 350° C., or from about 305° C. to about 325° C.); the activation temperature may be at least about 310° C. (e.g., from about 310° C. to about 400° C., from about 310° C. to about 375° C., from about 310° C. to about 350° C., or from about 310° C. to about 325° C.); the activation temperature may be at least about 315° C. (e.g., from about 315° C. to about 400° C., from about 315° C. to about 375° C., from about 315° C. to about 350° C., or from about 315° C. to about 325° C.); the activation temperature may be at least about 320° C. (e.g., from about 320° C. to about 400° C., from about 320° C. to about 375° C., or from about 320° C. to about 350° C.), or the activation temperature may be at least about 325° C. (e.g., from about 325° C. to about 400° C., from about 325° C. to about 375° C., or from about 325° C. to about 350° C.).

Following activation, such as according to step (a) described above, a subsequent operating step is performed, such as according to step (b) described above, in which a synthesis gas comprising H₂ and CO is contacted with the LPG synthesis catalyst system at an initial operation temperature. Importantly, whereas the initial operation temperature is less than the activation temperature of step (a), an advantageous level of performance (e.g., selectivity to and/or yield of LPG hydrocarbons) may nonetheless be attained in view of effects of the preceding activation on the LPG synthesis catalyst system (e.g., by establishing a hydrocarbon pool). Therefore, between an activation step (a) and an operating step (b), processes may comprise reducing the temperature of the LPG synthesis catalyst system from the activation temperature to the initial operation temperature. The temperature reduction may coincide with suspending contacting with the activating gas, used in step (a), and initiating contacting with the synthesis gas, used in step (b). However, various ways of transitioning between the activating gas/activation temperature requirements of step (a) and the synthesis gas/initial operation temperature requirements of step (b) will be apparent to those skilled in the art having knowledge of the present disclosure. Suspending contacting of the activating gas and initiating contacting with the synthesis gas may occur prior to, during, or subsequent to, reducing the temperature. For example, the composition of the gas flowing to the LPG synthesis catalyst system may be changed abruptly from that of the activating gas to that of the synthesis gas at any of these time points relative to the time of reducing the temperature. Alternatively, these compositions may be changed gradually at any of these time points, or over a transition time encompassing the temperature reduction time, or within the temperature reduction time. According to some embodiments, in which the activating gas and the synthesis gas have the same composition, only a temperature reduction is required between step (a) and step (b), without a change in gas composition.

From the above description, it can be appreciated that, in some embodiments, a flow of gas (e.g., the activating gas or the synthesis gas, which may be the same) and/or an elevated temperature (e.g., the activation temperature, the initial operation temperature, or a temperature between these two) may be maintained between step (a) and step (b). For example, both a gas flow and an elevated temperature may be maintained. However, processes within the scope of the invention do not necessarily require the maintenance of such gas flow and/or such elevated temperature between these steps. For example, step (a) may result in an “activated” or “primed” catalyst system, following which step gas flow and/or elevated temperature (e.g., heating) may be suspended for a relatively short suspension period (e.g., at least about 1 minute, such as from about 1 minute to about 24 hours; at least about 30 minutes, such as from about 30 minutes to about 24 hours; at least about 2 hours, such as from about 2 hours to about 24 hours; at least about 8 hours, such as from about 8 hours to about 24 hours; or at least about 12 hours, such as from about 12 hours to about 24 hours) or for a relatively long suspension period (e.g., at least about 1 day, such as from about 1 day to about 720 days; at least about 3 days, such as from about 3 days to about 360 days; at least about 5 days, such as from about 5 days to about 270 days; at least about 10 days, such as from about 10 days to about 180 days; or at least about 30 days, such as from about 30 days to about 60 days). According to particular embodiments, any such suspension period, for example any such relatively long suspension period, may be a shutdown period in which both gas flow and elevated temperature (e.g., heating) are suspended.

Whether a suspension period or, more particularly, a shutdown period, occurs following step (a), gas flow and/or elevated temperature (e.g., heating) may be resumed following such period, as necessary to perform step (b), with the catalyst system now having been activated or primed in step (a) to attain advantages as described herein. In the case of a shutdown period, catalyst(s) of the catalyst system (e.g., alcohol synthesis catalyst, dehydration catalyst, or bifunctional catalyst) may be removed from vessel(s) used to perform step (a) (e.g., used to maintain catalyst(s) under conditions used in this step). To subsequently perform step (b), the activated catalyst(s), or activated catalyst system, may be returned to the same vessel(s) used to perform step (a), or otherwise such activated catalyst(s), or activated catalyst system, may be input (charged or loaded) to different vessel(s). For example, in practicing processes as described herein, step (a) may be performed in one facility, to obtain the activated catalyst(s), or activated catalyst system, and step (b) may be performed in a different facility, to convert the synthesis gas to LPG hydrocarbons.

Embodiments of the invention further extend to processes utilizing an LPG synthesis catalyst system, or catalyst(s) of such system, which has/have been already activated as described herein. Representative processes for producing an LPG product comprising propane and/or butane comprise: contacting a synthesis gas comprising H₂ and CO with an LPG synthesis catalyst system, as described herein, at an initial operation temperature, as also described herein, that is less than an activation temperature (i.e., performing a step corresponding to step (b) as described herein). The catalyst system in such embodiments is namely an activated catalyst system, having been contacted with an activating gas as described herein at the activation temperature or above (i.e., having been subjected to a step corresponding to step (a) as described herein). More broadly, in the case of an LPG synthesis catalyst system comprising both an alcohol synthesis catalyst and a dehydration catalyst, at least one of such catalysts, and preferably the dehydration catalyst, may have been contacted with an activating gas as described herein at the activation temperature or above. The activated catalyst system, or catalyst system in which at least one catalyst (e.g., the dehydration catalyst) has been contacted with an activating gas at the activation temperature or above, may be used in subsequent operation as described herein. The activated catalyst system, or catalyst system in which at least one catalyst (e.g., the dehydration catalyst) has been contacted with an activating gas at the activation temperature or above, may have been subjected to a pre-activation step as described herein.

In any of the embodiments described herein, subsequent operation (e.g., subsequent to step (b)) may include increasing temperature to subsequent operation temperatures above that of the initial operation temperature, such as to compensate for gradual losses of activity of the LPG synthesis catalyst system, while maintaining a given level of performance (e.g., selectivity to and/or yield of LPG hydrocarbons). In some embodiments, a subsequent operation temperature may exceed the activation temperature. In general, the use of an activation step (a), such as relative to a reference process excluding this step, improves overall stability of the LPG synthesis catalyst system, thereby prolonging its effective “life,” or time over which a given level of performance (e.g., selectivity to and/or yield of LPG hydrocarbons) may be maintained, before reaching a given “end-of-life” temperature and/or “end-of-life” coke (carbon) deposition level. Such end-of-life temperature or end-of-life coke deposition level may characterize the LPG synthesis catalyst system, in terms of requiring regeneration. For example, an end-of-life temperature may be any discrete temperature within the range generally from 325° C. to 500° C., typically from 325° C. to 450° C., and often from 325° C. to 400° C. An end-of-life coke (carbon) deposition level within the catalyst system, or on either an alcohol synthesis catalyst or a dehydration catalyst, may be, for example, any discrete amount generally from 1 wt-% to 25 wt-%, typically from 1 wt-% to 20 wt-%, and often from 1 wt-% to 15 wt-%.

Temperatures described herein, including the “activation temperature,” “initial operation temperature,” “subsequent operation temperature,” and/or “end-of-life temperature,” may refer to the inlet temperatures of the LPG synthesis catalyst system, namely the temperatures at which the activating gas or the synthesis gas (as the case may be, and which may have the same composition) first contact the LPG synthesis catalyst system, or component of this system (e.g., an alcohol synthesis catalyst in the case of such system comprising two separate catalyst types). Temperatures described herein, according to other embodiments, may refer to the peak or maximum temperature of, or within, the LPG synthesis catalyst system. In the case of the LPG catalyst system being contained in multiple reactors (e.g., an alcohol synthesis catalyst being contained in a first, upstream reactor and a dehydration catalyst being contained in a second, downstream reactor), such peak or maximum temperature may be the highest temperature of all temperatures in the multiple reactors. Temperatures described herein, according to yet other embodiments, may refer to the average temperature of the LPG synthesis catalyst system, which may be more specifically the weighted average bed temperature (WABT). This may be determined, for example, by measuring temperatures within the LPG synthesis catalyst system at multiple points, determining average values between adjacent points, and weighting those average values with weighting factors, totaling 100%, according to the weight percentage of catalyst of the LPG synthesis catalyst system represented by the average values. The weighted average values are then added. For example, the multiple points in the case of an axial flow reactor may be measured along the central axis within a bed of catalyst of the LPG synthesis catalyst system, to approximate WABT in the absence of radial temperature gradients. Notwithstanding the above explanation, the determination of WABT is readily understood by those skilled in the art. In the case of the LPG catalyst system being contained in multiple reactors (e.g., an alcohol synthesis catalyst being contained in a first, upstream reactor and a dehydration catalyst being contained in a second, downstream reactor), the WABT may be determined with respect to the entire amount of catalyst in the multiple reactors. Preferably, for comparative purposes, in the case of one temperature (e.g., the activation temperature) referring to one of the measurements described above, then one or more other temperatures (e.g., the initial operation temperature) also refers to this measure. For example, both the activation temperature and the initial operation temperature may be inlet temperatures of the LPG synthesis catalyst system, or otherwise both of these temperatures may be average temperatures, such as determined by their WABT.

Activation, by contacting an activating gas with an LPG synthesis catalyst system, may, according to some embodiments, require a combination of (i) the activation temperature (or threshold temperature) or above, with (ii) a time (or threshold time) at this activation temperature (or threshold temperature) or above, such as a combination of a threshold temperature and threshold time needed to establish a hydrocarbon pool. For example, in step (a), contacting with the activating gas (e.g., a synthesis gas comprising H₂ and CO) at the activation temperature may occur for an activation time, or activation period, of at least about 1 hour (e.g., from about 1 hour to about 100 hours, from about 1 hour to about 72 hours, from about 1 hour to about 48 hours, from about 1 hour to about 24 hours, from about 1 hour to about 12 hours, or from about 1 hour to about 6 hours); at least about 4 hours (e.g., from about 4 hours to about 100 hours, from about 4 hours to about 72 hours, from about 4 hours to about 48 hours, from about 4 hours to about 24 hours, or from about 4 hours to about 12 hours); at least about 8 hours (e.g., from about 8 hours to about 100 hours, from about 8 hours to about 72 hours, from about 8 hours to about 48 hours, or from about 8 hours to about 24 hours); or at least about 24 hours (e.g., from about 24 hours to about 100 hours, from about 24 hours to about 72 hours, or from about 24 hours to about 48 hours). It can therefore be appreciated that a given activation step (a) may be defined by more than a single activation temperature/activation time combination. For example, in the case of contacting the activating gas with the LPG synthesis catalyst system at a temperature of 300° C. for 3 hours, at a temperature of 315° C. for 2 hours, and at a temperature of 310° C. for 4 hours, this may be considered an activation step comprising contacting the activating gas with the LPG synthesis catalyst system at (i) an activation temperature of 300° C. or above for a time of 9 hours or above, (ii) an activation temperature of 310° C. or above for a time of 6 hours, or (iii) an activation temperature of 315° C. or above for a time of 2 hours. Therefore, in the case of an activation step characterized by (i), (ii), or (iii), a subsequent operating step, in which the LPG synthesis catalyst system is contacted with a synthesis gas comprising H₂ and CO, may be carried out at an initial operation temperature of (i) less than 300° C., (ii) less than 310° C., or (iii) less than 315° C.

In some exemplary embodiments, the activation temperature, regardless of the time for which this activation temperature or above is utilized in the activation step, is at least 300° C., whereas the initial operation temperature is less than 300° C. In other exemplary embodiments, the activation temperature, regardless of its value or the time for which this activation temperature or above is utilized in the activation step, may be at least about 5° C. above (e.g., from about 5° C. to about 25° C. above, or from about 5° C. to about 50° C. above); at least about 10° C. above (e.g., from about 10° C. to about 25° C. above, or from about 10° C. to about 50° C. above); at least about 15° C. above (e.g., from about 15° C. to about 25° C. above, or from about 15° C. to about 50° C. above); or at least about 20° C. above (e.g., from about 25° C. to about 50° C. above); the initial operation temperature. That is, the initial operation temperature may be less than the activation temperature by a value within any of these ranges (e.g., the initial operation may be at least 5° C. less than the activation temperature). Representative processes may comprise an activation step (a) and an operating step (b), with particular embodiments utilizing any combinations of activation temperatures, initial operation temperatures, differences between these temperatures, activation times, and end-of-life conditions (temperature and/or carbon deposition levels) as described above.

According to other embodiments, an activation step (a) may be performed to establish a given yield, or threshold yield, of LPG hydrocarbons, as a basis for proceeding to operation step (b). For example, in representative processes, optionally in combination with any of the activation temperatures, initial operation temperatures, differences between these temperatures, activation times, and/or end-of-life conditions (temperature and/or carbon deposition levels) as described above, step (a) may be carried out for an activation period sufficient to obtain an activation LPG hydrocarbon yield that is at least a given threshold LPG hydrocarbon yield. Such threshold LPG hydrocarbon yield may be, for example, about 10%, about 15%, about 20%, about 25%, or about 30%. The threshold LPG hydrocarbon yield may be as defined herein, according to the per-pass (or single pass) yield of LPG hydrocarbons (e.g., product of the per-pass CO conversion and the carbon selectivity to LPG hydrocarbons). Therefore, a threshold LPG hydrocarbon yield may be the basis for the extent to which an activation step (a) is performed, at a given activation temperature. In other embodiments, a combination of a threshold LPG hydrocarbon yield and a time over which this yield is obtained, such as any activation time, or activation period, as described herein, may be the basis for the extent to which activation step (a) is performed, according to particular processes. For example, an activation LPG hydrocarbon yield of at least about 20% for a time of at least about 4 hours may characterize a given activation step. In still other embodiments, the combination of a threshold LPG hydrocarbon yield and a time may refer to any of these parameters as described above, in the alternative, such as in the case of activation step (a) being carried out for an activation period sufficient to obtain an activation LPG hydrocarbon yield of at least about 20%, or for an activation period of at least about 4 hours (e.g., whichever occurs first), to characterize a given process.

In representative processes, optionally in combination with any of the activation temperatures, initial operation temperatures, differences between these temperatures, activation times, threshold LPG hydrocarbon yields (for activation and/or initial operation), and/or end-of-life conditions (temperature and/or carbon deposition levels) as described above, initial operation (or simply operating) step (b) may be carried out at an initial operation temperature (e.g., as described herein), sufficient to obtain an initial operation LPG hydrocarbon yield, which may correspond to at least any threshold LPG hydrocarbon yield as described above with respect to activation step (a). The initial operation LPG hydrocarbon yield may be the same as, or different from, the activation LPG hydrocarbon yield. Therefore, for example, optionally in combination with carrying out step (a) for an activation period sufficient to obtain a given activation LPG hydrocarbon yield as described above (e.g., at least about 20%), an initial operation LPG hydrocarbon yield in step (b) at the initial operation temperature may be at least about 10%, at least about 15%, at least about 20%, at least about 25%, or at least about 30%.

According to some embodiments, the activation temperature may be defined by a minimum temperature needed to obtain a given activation LPG hydrocarbon yield as described above (e.g., at least about 20%), and/or the initial operation temperature may, in the same manner, be defined by a minimum temperature needed to obtain a given initial operation LPG hydrocarbon yield as described above (e.g., at least about 20%). In some cases, for example, representative process may comprise, prior to activation step (a), contacting the activating gas with the LPG synthesis catalyst system at a temperature that is below the activation temperature and that is not sufficient to obtain the activation LPG hydrocarbon yield, and then increasing the LPG synthesis catalyst system temperature to the activation temperature, which is sufficient, or determined to be sufficient (e.g., following gradual or stepwise temperature increases, with corresponding determinations of LPG hydrocarbon yield), to obtain the activation LPG hydrocarbon yield. In such cases, any temperature below that which is determined necessary to obtain the activation LPG hydrocarbon yield, may be referred to as a “pre-activation temperature.” Pre-activation, prior to activation step (a), may result in a significant yield of methanol and/or DME, as opposed to LPG hydrocarbons, with such yields being defined in a manner analogous to the yield of LPG hydrocarbons, as defined herein (e.g., determined as the product of the per-pass CO conversion and the carbon selectivity to methanol and/or DME). Pre-activation methanol and/or DME yields may independently have the same values, such as at least about 10%, at least about 15%, at least about 20%, at least about 25%, or at least about 30%, as described above with respect to activation LPG hydrocarbon yields and initial operation LPG hydrocarbon yield. Pre-activation may be performed over any suitable pre-activation time or pre-activation period, including any time or period as described above with respect to the activation time or activation period.

For example, representative processes may comprise, prior to activation step (a), contacting the activating gas (e.g., a synthesis gas comprising H₂ and CO) with the LPG synthesis catalyst system at a pre-activation temperature (of this catalyst system), sufficient to obtain a pre-activation methanol and/or DME yield as described herein (e.g., at least about 20%). Such pre-activation step may be followed by an activation step (a) as described herein. For example, following this pre-activation step, a representative process may comprise (a) increasing the LPG synthesis catalyst system temperature to an activation temperature (e.g., above the pre-activation temperature) or above, sufficient to provide an activation LPG hydrocarbon yield as described herein (e.g., at least about 30%). As an alternative to activation LPG hydrocarbon yield, or in combination with this parameter, such activation step (a) may be carried out with any other parameter(s) described herein (e.g., activation temperature and/or activation time), and the process itself may likewise be carried out with any other parameter(s) described herein (e.g., initial operation temperature, difference between activation and initial operation temperature, threshold LPG hydrocarbon yields (for activation and/or initial operation), and/or end-of-life conditions (temperature and/or carbon deposition levels)). Following activation step (a), representative processes may comprise, as an initial operation step (b), decreasing the LPG synthesis catalyst system temperature to an initial operation temperature, sufficient to maintain an initial operation LPG hydrocarbon yield as described herein (e.g., at least about 30%). Preferably, according to any process as described herein, a flow of the activating gas (e.g., a synthesis gas comprising H₂ and CO) is maintained during activation step (a) and initial operation step (b), as well as during any pre-activation step as described herein.

Advantageously, according to any process as described herein, the use of activation step (a) may confer unexpected performance advantages, which may be characterized or quantified in terms performance relative to a reference process, or comparative process, carried out in the absence of activation step (a). For example, in processes described herein that utilize a pre-activation step, the pre-activation temperature and the initial operation temperature may be the same, such that in some cases, the only difference between pre-activation and initial operation is that the former is conducted prior to activation step (a) and the latter is conducted subsequent to activation step (a). The effect of the activation step (a) may reside in a significant increase in LPG hydrocarbon yield occurring in the latter (initial operation) but not in the former (pre-activation), which increase would be considered surprising in view of the general expectation that the same or similar processing conditions should result in the same or similar performance.

In general, the effect of activation step (a), in processes described herein, may be validated against reference processes that are the same in all respects (e.g., utilize the same conditions in terms of temperature, pressure, WHSV, synthesis gas and catalyst compositions, etc.) but lack this activation step. For example, in a reference process consisting of initial operation step (b) in the absence of activation step (a), a reference initial LPG hydrocarbon yield (at the initial operation temperature) may be less than an initial operation LPG hydrocarbon yield in step (b) of the process described herein, which includes activation step (a). The initial operation LPG hydrocarbon yield may be as described herein (e.g., at least about 20%), whereas the reference initial LPG hydrocarbon yield may be less than about 10%, less than about 5%, less than about 3%, or even less than about 1%. In addition to, or alternatively to, the reference process being characterized by a comparatively low reference initial LPG hydrocarbon yield, this reference process may be characterized by a comparatively high reference initial methanol and/or DME yield. For example, in a reference process consisting of initial operation step (b) in the absence of activation step (a), a reference initial methanol yield and/or reference initial DME yield (at the initial operation temperature) may be greater than a respective, initial operation methanol yield and/or initial operation DME yield in step (b) of the process described herein, which includes activation step (a). The initial operation methanol yield and/or initial operation DME yield may be may be less than about 10%, less than about 5%, less than about 3%, or even less than about 1% (as represented by the methanol yield or the DME yield, or otherwise these yields in combination), whereas the reference initial methanol yield and/or reference initial DME yield may be as described herein with respect to a pre-activation methanol and/or DME yield (as represented by the methanol yield or the DME yield, or otherwise these yields in combination). Therefore, the reference initial methanol yield and/or reference initial DME yield may be, for example, as at least about 10%, at least about 15%, at least about 20%, at least about 25%, or at least about 30%.

Likewise, the beneficial effect of the combination of activation step (a) at a relatively high activation temperature with initial operation step (b) at a relatively low initial operation temperature may be validated against reference processes that are the same in all respects (e.g., utilize the same conditions in terms of temperature, pressure, WHSV, synthesis gas and catalyst compositions, etc.) but lack the initial operation step. For example, in a reference process consisting of activation step (a), such as a reference process consisting of contacting the activating gas (e.g., a synthesis gas comprising H₂ and CO) with the LPG synthesis catalyst system at the activation temperature of a process described herein, but in the absence of reducing the temperature to the initial operation temperature of step (b), a reference deactivation rate of the LPG synthesis catalyst system may be greater than an operating deactivation rate of the process described herein, obtained from step (a) being performed in combination with step (b). In the case of processes described herein that include a pre-activation step, in a reference process consisting of the pre-activation step and step (a), such as a reference process consisting of contacting the activating gas (e.g., a synthesis gas comprising H₂ and CO) with the LPG synthesis catalyst system at the pre-activation temperature and at the activation temperature, but in the absence of reducing the temperature to the initial operation temperature of step (b), a reference deactivation rate of the LPG synthesis catalyst system may be greater than an operating deactivation rate of the process described herein, obtained from the pre-activation step and step (a) being performed in combination with step (b). An increased deactivation rate of the LPG synthesis catalyst system corresponds to reduced stability and therefore generally higher costs associated with, among other deleterious effects, more frequent catalyst replacement. The deactivation rate may be quantified, for example, according to the rate of increase in the LPG synthesis catalyst system temperature (e.g., inlet temperature, peak temperature, or WABT as described herein) needed to maintain a given performance parameter (e.g., CO conversion, or otherwise selectivity to and/or yield of LPG hydrocarbons), or otherwise according to the rate of loss in any such performance parameter, at a given LPG synthesis catalyst system temperature.

An LPG synthesis catalyst system may comprise two or more different catalyst types, or a single catalyst having two or more different types of functional constituents. The different catalyst types or single catalyst may be contained in one or more LPG synthesis reactors (e.g., in a series or parallel arrangement), at least one of which is fed a synthesis gas comprising H₂ and CO, such as during an operating step (b), for contacting with the LPG synthesis catalyst system, or at least one catalyst type of the system. Preferably, the different catalyst types or single catalyst are contained within a single LPG synthesis reactor, but it is also possible, for example, for separate LPG synthesis reactors to contain each of the different catalyst types. It is also possible for separate LPG synthesis reactors to contain the different catalyst types at different weight ratios and/or in different bed configurations. In one embodiment, a first (upstream) LPG synthesis reactor (e.g., a methanol synthesis reactor) may contain an alcohol synthesis catalyst (e.g., methanol synthesis catalyst) as described herein, and a second (downstream) LPG synthesis reactor (e.g., a dehydration reactor) may contain a dehydration catalyst as described herein. The use of separate reactors allows for reaction conditions to be more precisely aligned with different stages of reactions used to carry out the synthesis of LPG hydrocarbons from a synthesis gas. In general, different catalyst types or a single catalyst may be utilized in any particular bed configuration (e.g., fixed bed or fluidized bed), or, in the case of different catalyst types in a fixed bed configuration, in any particular arrangement of individual beds of one catalyst type or another, such as in the case of using one or more beds an alcohol synthesis catalyst (e.g., methanol synthesis catalyst) alone, one or more beds of a dehydration catalyst alone, one or more beds of a mixture of catalyst types at a selected mixing ratio or differing mixing ratios, and/or combinations of such beds. Regardless of the particular bed configuration or particular arrangement of individual beds, preferably the catalyst types or single catalyst is/are in the form of discreet particles, as opposed to a monolithic form of catalyst. For example, such discreet particles of an alcohol synthesis catalyst (e.g., methanol synthesis catalyst), a dehydration catalyst, or a bi-functional catalyst may have a spherical or cylindrical diameter of less than about 10 mm and often less than about 5 mm (e.g., about 2 mm). In the case of cylindrical catalyst particles (e.g., formed by extrusion), these may have a comparable length dimension (e.g., from about 1 mm to about 10 mm, such as about 5 mm).

LPG synthesis catalyst systems may, more particularly, comprise at least two components having different catalytic activities, with such components either being (a) separate compositions (e.g., each composition being in the form of separate particles) of an alcohol synthesis catalyst (e.g., a methanol synthesis catalyst) and a dehydration catalyst, or (b) functional constituents of a bi-functional catalyst (e.g., the catalyst being in the form of separate particles) that is a single composition having both an alcohol synthesis-functional constituent (e.g., a methanol synthesis-functional constituent) and a dehydration-functional constituent. As noted above, a dehydration catalyst may alternatively be referred to as an alcohol to LPG hydrocarbon conversion (ATLPG) catalyst, such as a methanol to LPG hydrocarbon conversion (MTLPG) catalyst, and a dehydration-functional constituent may alternatively be referred to as an alcohol to LPG hydrocarbon conversion-(ATLPG-) functional constituent, such as a methanol to LPG hydrocarbon conversion-(MTLPG-) functional constituent.

The separate catalyst compositions, or otherwise the functional constituents of a bi-functional catalyst, may be present in equal or substantially equal weight ratios. For example, the (i) alcohol synthesis catalyst (e.g., methanol synthesis catalyst) and (ii) dehydration catalyst may be present in the catalyst mixture in a weight ratio of (i):(ii) of about 1:1. Otherwise, the (i) alcohol synthesis-functional constituent (e.g., methanol synthesis-functional constituent) and (ii) dehydration-functional constituent may be present in the bi-functional catalyst in a weight ratio of (i):(ii) of about 1:1. Generally, however, these weight ratios may vary, for example the weight ratios of (i):(ii) in each case may be from about 10:1 to about 1:10, such as from about 5:1 to about 1:5, or from about 3:1 to about 1:3.

In addition to such separate compositions of catalysts or single composition of a bi-functional catalyst, representative LPG synthesis catalyst systems may further comprise additional components, e.g., particles of silicon carbide, silicon nitride, silica or sand, acting to absorb heat and/or alter the distribution of solids. Such additional components may be present in an amount, for example, of at least about 10 wt-% (e.g., from about 10 wt-% to about 80 wt-%), at least about 20 wt-% (e.g., from about 20 wt-% to about 70 wt-%), or at least about 40 wt-% (e.g., from about 40 wt-% to about 60 wt-%), of a given catalyst system. Such additional components may therefore substantially lack catalytic activity and serve non-catalytic purposes. Alternatively, or in combination, additional components may include additional compositions having catalytic activity and/or additional functional constituents having catalytic activity. For example, representative LPG synthesis catalyst systems may comprise additional compositions as described above, such as an additional composition comprising a stabilizer, which may be a noble metal stabilizer such as platinum or a non-noble metal stabilizer such as yttrium (e.g., in elemental form, in the form of an oxide such as yttria or other form) and/or an additional composition comprising one or more promoters selected from the group consisting of Mn, Mg, and Si (e.g., independently in elemental forms, oxide forms, or other forms). In this regard, it can be appreciated that a catalyst system comprising an alcohol synthesis catalyst such as a methanol synthesis catalyst and a dehydration catalyst is not meant to preclude the presence of other catalysts. Likewise, the term “bi-functional catalyst” is not meant to preclude the presence of additional functional constituents. In some embodiments, however, an LPG synthesis catalyst system may consist of, or consist essentially of, two different catalyst types, or otherwise a single catalyst of such catalyst system may consist of, or consist essentially of, two different types of functional constituents. An LPG synthesis catalyst system may also consist of, or consist essentially of, a single type of bi-functional catalyst.

A representative alcohol synthesis catalyst (e.g., methanol synthesis catalyst) or alcohol synthesis-functional constituent (e.g., methanol synthesis-functional constituent) of a bi-functional catalyst may comprise one or more alcohol synthesis-active metals (e.g., methanol synthesis-active metals), with representative metals being selected from the group consisting of copper (Cu), zinc (Zn), aluminum (Al), platinum (Pt), palladium (Pd), and chromium (Cr). In the case of such alcohol (e.g., methanol) synthesis-active metals being Pt and/or Pd, these may, in addition to being considered alcohol (e.g., methanol) synthesis-active metals, also be considered noble metal stabilizers. Any alcohol synthesis-active metals may be in their elemental forms or compound forms. For example, in the case of Cu, Pt, and Pd, these metals are preferably in their elemental forms and, in the case of Zn, Al, and Cr, these metals are preferably in their oxide forms, namely ZnO, Al₂O₃, and Cr₂O₃, respectively. In some preferred embodiments, all or a portion of Cu, in case of an alcohol synthesis catalyst (e.g., a methanol synthesis catalyst) or alcohol synthesis-functional constituent (e.g., methanol synthesis-functional constituent) comprising this metal, may be in its oxide form CuO. A particular representative alcohol synthesis catalyst, which may more particularly be a methanol synthesis catalyst, is a copper and zinc oxide on alumina catalyst, comprising or consisting essentially of Cu/ZnO/Al₂O₃. Such “CZA” alcohol synthesis catalyst (e.g., methanol synthesis catalyst) may also be an alcohol synthesis-functional constituent (e.g., methanol synthesis-functional constituent) of a bi-functional catalyst.

In the case of an alcohol synthesis catalyst (e.g., methanol synthesis catalyst) or alcohol synthesis-functional constituent (e.g., methanol synthesis-functional constituent) of a bi-functional catalyst, the alcohol synthesis-active metals (e.g., methanol synthesis-active metals) Cu, Zn, Pt, Pd, and/or Cr, particularly when in their elemental forms, may be supported on a solid support. Representative solid supports comprise one or more metal oxides, for example those selected from the group consisting of aluminum oxide, silicon oxide, titanium oxide, zirconium oxide, magnesium oxide, calcium oxide, iron oxide, vanadium oxide, chromium oxide, nickel oxide, tungsten oxide, and strontium oxide. The phrase “on a solid support” is intended to encompass alcohol synthesis catalyst solid supports (e.g., methanol synthesis catalyst solid supports) and bi-functional catalyst solid supports in which the alcohol synthesis-active metal(s) (e.g., methanol synthesis-active metal(s)) is/are on the support surface and/or within a porous internal structure of the support. Specific examples of alcohol synthesis catalysts, such as methanol synthesis catalysts, or alcohol synthesis-functional constituents, such as methanol synthesis-functional constituents, therefore include Pd that is supported on a solid support of a metal oxide (e.g., aluminum oxide) and present in the catalyst or constituent in an amount as described herein.

For an alcohol synthesis catalyst (e.g., methanol synthesis catalyst) or an alcohol synthesis-functional constituent (e.g., methanol synthesis-functional constituent) comprising one or more of Cu, Zn, Al, Pt, Pd, and Cr, regardless of their particular form(s), such metal(s) may be present independently in an amount, in the respective alcohol synthesis catalyst (e.g., methanol synthesis catalyst) or bi-functional catalyst, generally from about 0.5 wt-% to about 45 wt-%, typically from about 1 wt-% to about 20 wt-%, and often from about 1 wt-% to about 10 wt-%, relative to the weight of the alcohol synthesis catalyst alone or the alcohol synthesis-functional constituent alone, or possibly relative to a bi-functional catalyst as a whole. In some embodiments, the metal Cu may be present, in an alcohol synthesis catalyst or bi-functional catalyst, in an amount from about 1 wt-% to about 25 wt-%, such as from about 1 wt-% to about 15 wt-%, relative to the weight of the alcohol synthesis catalyst alone or the alcohol synthesis-functional constituent alone, or possibly relative to a bi-functional catalyst as a whole. Independently or in combination with such amounts of Cu, the metal Zn may be present, in an alcohol synthesis catalyst such as a methanol synthesis catalyst, or bi-functional catalyst, in an amount from about 1 wt-% to about 20 wt-%, such as from about 1 wt-% to about 10 wt-%, relative to the weight of the alcohol synthesis catalyst alone or the alcohol synthesis-functional constituent alone, or possibly relative to a bi-functional catalyst as a whole. Independently or in combination with such amounts of Cu and/or Zn, the metal Al may be present, in an alcohol synthesis catalyst such as a methanol synthesis catalyst, or bi-functional catalyst, in an amount from about 1 wt-% to about 30 wt-%, such as from about 5 wt-% to about 20 wt-%, relative to the weight of the alcohol synthesis catalyst alone or the alcohol synthesis-functional constituent alone, or possibly relative to a bi-functional catalyst as a whole. Independently or in combination with such amounts of Cu, Zn, and/or Al, any one or more of the metals Pt, Pd, and/or Cr may be present, in an alcohol synthesis catalyst (e.g., methanol synthesis catalyst) or bi-functional catalyst, independently in an amount, or in a combined amount, from about 0.5 wt-% to about 10 wt-%, such as from about 1 wt-% to about 5 wt-%, relative to the weight of the alcohol synthesis catalyst alone or the alcohol synthesis-functional constituent alone, or possibly relative to a bi-functional catalyst as a whole.

The alcohol synthesis catalyst (e.g., methanol synthesis catalyst) or a methanol synthesis-functional constituent may further comprise a stabilizer, such as a noble metal stabilizer (e.g., platinum) or a non-noble metal stabilizer such as yttrium (e.g., in elemental form, in the form of an oxide such as yttria or other form) and/or one or more promoters selected from the group consisting of Mn, Mg, and/or Si (e.g., independently in elemental forms, oxide forms, or other forms), in respective amounts as described above. In the case of an alcohol synthesis catalyst (e.g., methanol synthesis catalyst) or alcohol synthesis-functional constituent (e.g., methanol synthesis-functional constituent) of a bi-functional catalyst, the alcohol synthesis-active metal(s) (e.g., methanol synthesis-active metal(s)), or any forms of such metals (e.g., their respective oxide forms), and optionally any solid support, may constitute all or substantially all of the catalyst or functional constituent. For example, the alcohol synthesis-active metal(s) (e.g., methanol synthesis-active metal(s)), or any forms of such metals, and optionally any solid support, may be present in a combined amount representing at least about 90%, at least about 95%, or at least about 99%, of the total weight of the alcohol synthesis catalyst (e.g., methanol synthesis catalyst) or alcohol synthesis-functional constituent (e.g., methanol synthesis-functional constituent). In the case of an alcohol synthesis catalyst or an alcohol synthesis-functional constituent further comprising one or more stabilizers and/or one or more promoters of Mn, Mg, and/or Si, the alcohol synthesis-active metal(s), or any forms of such metals, and optionally any solid support, together with the stabilizer(s) or any forms of the stabilizer(s) (e.g., platinum, yttrium, or any of their respective forms) and/or the promoter(s) or any forms of the promoter(s), may be present in a combined amount representing at least about 90%, at least about 95%, or at least about 99%, of the total weight of the alcohol synthesis catalyst (e.g., methanol synthesis catalyst) or alcohol synthesis-functional constituent (e.g., methanol synthesis-functional constituent).

In a representative alcohol synthesis catalyst (e.g., methanol synthesis catalyst) or bi-functional catalyst, any metal(s) other than Cu, Zn, Al, Pt, Pd, and/or Cr may be present in minor amounts, may be substantially absent, or may be absent. For example, any such other metal(s) may be independently present in an amount of less than about 1 wt-%, less than about 0.1 wt-%, or even less than about 0.05 wt-%, based on the total catalyst weight. Alternatively, any two or more of such other metals may be present in a combined amount of less than about 2 wt-%, less than about 0.5 wt-%, or even less than about 0.1 wt-%, based on the total catalyst weight. According to particular embodiments, for example in the case of (i) an alcohol synthesis catalyst such as a methanol synthesis catalyst comprising a solid support, or (ii) a bi-functional catalyst comprising, as a dehydration-functional constituent, a zeolite or non-zeolitic molecular sieve, such metals other than Cu, Zn, Al, Pt, Pd, and/or Cr, and present in the amounts described above, may be, more particularly, (a) metal(s) other than Cu, Zn, Al, Pt, Pd, Cr, and Si; metal(s) other than Cu, Zn, Al, Pt, Pd, Cr, Si, Ti, Zr, Mg, Ca, and Sr; or metal(s) other than Cu, Zn, Al, Pt, Pd, Cr, Si, Ti, Zr, Mg, Ca, Sr, and Y, (b) metal(s) other than Cu, Zn, Al, Pt, Pd, Cr, Si, and P; metal(s) other than Cu, Zn, Al, Pt, Pd, Cr, Si, P, Mg, Zn, Fe, Co, Ni, and Mn; or metal(s) other than Cu, Zn, Al, Pt, Pd, Cr, Si, P, Mg, Zn, Fe, Co, Ni, Mn, and Y, or (c) metal(s) other than Cu, Zn, Al, Pt, Pd, Cr, and Y; metal(s) other than Cu, Zn, Al, Pt, Pd, Cr, Mn, Mg, and Si; or metal(s) other than Cu, Zn, Al, Pt, Pd, Cr, Y, Mn, Mg, and Si. For convenience, in these particular embodiments, Si will be considered a “metal” in terms of its contribution to an alcohol synthesis catalyst, such as a methanol synthesis catalyst, or bi-functional catalyst.

A representative dehydration catalyst or dehydration-functional constituent of a bi-functional catalyst may comprise a zeolite (zeolitic molecular sieve) or a non-zeolitic molecular sieve (zeotype). Particular zeolites or non-zeolitic molecular sieves may have a structure type selected from the group consisting of CHA, TON, FAU, FER, BEA, ERI, MFI, MEL, MTW, MWW, MOR, LTL, LTA, EMT, MAZ, MEI, AFI, and AEI, and preferably selected from one or more of CHA, TON, FAU, FER, BEA, ERI, MFI, MOR, and MEI. The structures of zeolites having these and other structure types are described, and further references are provided, in Meier, W. M, et al., Atlas of Zeolite Structure Types, 4^{th} Ed., Elsevier: Boston (1996). Specific examples include SSZ-13 (CHA structure), zeolite Y (FAU structure), zeolite X (FAU structure), MCM-22 (MWW structure), zeolite beta (BEA structure), ZSM-5 (MFI structure), and ZSM-22 (TON structure), with zeolite beta and ZSM-5 being exemplary.

Non-zeolitic molecular sieves (zeotypes) include ELAPO molecular sieves which are embraced by an empirical chemical composition, on an anhydrous basis, expressed by the formula:

(ELₓAl_{y}P_{z})O₂

wherein EL is an element selected from the group consisting of silicon, magnesium, zinc, iron, cobalt, nickel, manganese, chromium and mixtures thereof, x is the mole fraction of EL and is often at least 0.005, y is the mole fraction of aluminum and is at least 0.01, z is the mole fraction of phosphorous and is at least 0.01 and x+y+z=1. When EL is a mixture of metals, x represents the total mole fraction of such metals present. The preparation of various ELAPO molecular sieves is known, and examples of synthesis procedures and their end products may be found in U.S. Pat. No. 5,191,141 (ELAPO); U.S. Pat. No. 4,554,143 (FeAPO); U.S. Pat. No. 4,440,871 (SAPO); U.S. Pat. No. 4,853,197 (MAPO, MnAPO, ZnAPO, CoAPO); U.S. Pat. No. 4,793,984 (CAPO); U.S. Pat. Nos. 4,752,651 and 4,310,440. Preferred ELAPO molecular sieves are SAPO and ALPO molecular sieves. Generally, the ELAPO molecular sieves are synthesized by hydrothermal crystallization from a reaction mixture containing reactive sources of EL, aluminum, phosphorus and a templating agent. Reactive sources of EL are the metal salts of EL elements defined above, such as their chloride or nitrate salts. When EL is silicon, a preferred source is fumed, colloidal or precipitated silica. Preferred reactive sources of aluminum and phosphorus are pseudo-boehmite alumina and phosphoric acid. Preferred templating agents are amines and quaternary ammonium compounds. An especially preferred templating agent is tetraethylammonium hydroxide (TEAOH).

A particularly preferred dehydration catalyst or dehydration-functional constituent comprises an ELAPO molecular sieve in which EL is silicon, with such molecular sieve being referred to in the art as a SAPO (silicoaluminophosphate) molecular sieve. In addition to those described in U.S. Pat. Nos. 4,440,871 and 5,191,141, noted above, other SAPO molecular sieves that may be used are described in U.S. Pat. No. 5,126,308. Of the specific crystallographic structures described in U.S. Pat. No. 4,440,871, SAPO-34, i.e., structure type 34, represents a preferred component of an LPG synthesis catalyst system. The SAPO-34 structure (CHA structure) is characterized in that it adsorbs xenon but does not adsorb iso-butane, indicating that it has a pore opening of about 4.2 Å. Accordingly, a representative dehydration catalyst or dehydration-functional constituent of a bi-functional catalyst may comprise SAPO-34 or other SAPO molecular sieve, such as SAPO-17, which is likewise disclosed in U.S. Pat. No. 4,440,871 and has a structure characterized in that it adsorbs oxygen, hexane, and water but does not adsorb iso-butane, indicative of a pore opening of greater than about 4.3 Å and less than about 5.0 Å. Due to its acidity, SAPO-34 can catalyze the conversion of an alcohol intermediate, such as a methanol intermediate, to olefins such as propylene. Without being bound by theory, it is believed that the characteristic hydrogen partial pressures used in the LPG synthesis stage not only promote the hydrogenation of these olefins, but also stabilize the dehydration catalyst/functional constituent by preventing coking. According to particular embodiments, the dehydration catalyst or dehydration-functional constituent may comprise a zeolite (zeolitic molecular sieve) of ZSM-5 or SSZ-13 or a non-zeolitic molecular sieve (zeotype) of SAPO-34 or SAPO-17. With respect to any particular zeolite or non-zeolitic molecular sieve that is used in an LPG synthesis catalyst system described herein, this may be present in any form according to which ion exchange sites are in their hydrogen form or otherwise exchanged with a suitable cation, non-limiting examples of which are cations of alkali metals (e.g., Na⁺), cations of alkaline earth metals (e.g., Ca⁺²), and ammonium cation (NH₄⁺). For example, as a zeolite, hydrogen form SSZ-13 (HSSZ-13) may be used; as a non-zeolitic molecular sieve, hydrogen form SAPO-34 (HSAPO-34) may be used.

In the case of the dehydration catalyst or dehydration-functional constituent comprising a zeolite or a non-zeolitic molecular sieve, such catalyst or functional constituent may be more particularly defined as a solid acid dehydration catalyst or solid acid dehydration-functional constituent, on the basis of the acidity exhibited by the zeolite or non-zeolitic molecular sieve. The acidity of a given zeolite or non-zeolitic molecular sieve may be determined, for example, by temperature programmed desorption (TPD) of a quantity of ammonia (ammonia TPD), from an ammonia-saturated sample of the material, over a temperature from 275° C. (527° F.) to 500° C. (932° F.), which is beyond the temperature at which the ammonia is physisorbed. The quantity of acid sites, in units of micromoles of acid sites per gram (μmol/g) of material, therefore corresponds to the number of micromoles of ammonia that is desorbed per gram of material in this temperature range. Alternatively, acidity may be calculated from, or based on, framework cation concentration of the zeolite or non-zeolitic molecular sieve. For example, in the particular case of the zeolite silicalite having a framework silica to alumina (SiO₂/Al₂O₃) molar framework ratio of 2000:1 (i.e., an Si/Al molar ratio of 1000:1), this would correspond to 16.6 μmol/g of acid sites, on the basis of the concentration of Al⁺³ cations. According to the TPD analysis above, in the absence of oligomerization, one NH₃ molecule would theoretically be absorbed per acid site or Al⁺³ cation in the above example. A representative zeolitic or non-zeolitic molecular sieve, or otherwise a representative dehydration catalyst or dehydration-functional constituent, has at least about 15 μmol/g (e.g., from about 15 to about 75 μmol/g) of acid sites, or at least about 25 μmol/g (e.g., from about 25 to about 65 μmol/g) of acid sites, measured by ammonia TPD or otherwise based on framework cation concentration. As noted above, in the case of zeolitic molecular sieves, acidity is a function of the silica to alumina (SiO₂/Al₂O₃) molar framework ratio, and, in embodiments in which the dehydration catalyst or dehydration-functional constituent comprises a zeolitic molecular sieve, its silica to alumina molar framework ratio may be less than about 2400 (e.g., from about 1 to about 2400), less than about 1000 (e.g., from about 1 to about 1000), less than about 400 (e.g., from about 1 to about 400), less than about 60 (e.g., from about 1 to about 60), or less than about 40 (e.g., from about 5 to about 40).

According to preferred embodiments, a dehydration catalyst (ATLPG catalyst, such as an MTLPG catalyst) or a dehydration-functional constituent (ATLPG-functional constituent, such as an MTLPG-functional constituent) may comprise one or more stabilizers such as a noble metal (e.g., platinum) or a non-noble metal (e.g., yttrium) in elemental form, in the form of an oxide or other form on a solid acid support comprising a zeolite or non-zeolitic molecular sieve. For example, the stabilizer(s) (e.g., platinum and/or yttrium) in elemental form or in a compound form may be dispersed uniformly or non-uniformly on such solid acid support. In the case of the dehydration catalyst or dehydration-functional constituent comprising a zeolite or a non-zeolitic molecular sieve, a stabilizer may be present in ion-exchange sites thereof, i.e., the dehydration catalyst or dehydration-functional constituent may comprise an ion-exchanged zeolite or an ion-exchanged non-zeolitic molecular sieve, having been prepared by ion-exchange to achieve a desired distribution of the stabilizer(s), such as a preferred distribution of a noble metal (e.g., platinum), within the zeolite or non-zeolitic molecular sieve. Regardless of the particular manner in which they are distributed, the stabilizer(s) (e.g., platinum and/or yttrium) may be present in such ATLPG catalyst (e.g., MTLPG catalyst) or ATLPG-functional constituent (e.g., MTLPG-functional constituent) in an amount, or a combined amount, as described herein, such as from about 0.03 wt-% to about 15 wt-%, based on the weight of the stabilizer(s), relative to the weight of the catalyst or functional constituent. Optionally, such catalyst or functional constituent may further comprise one or more promoters selected from the group consisting of manganese (Mn), magnesium (Mg), and silicon (Si), with these promoter(s) being independently in elemental form or a compound form, for example in their respective oxide forms of manganese oxide (MnO₂), magnesium oxide (MgO), and/or silica (SiO₂).

Other than zeolitic and/or non-zeolitic molecular sieves, representative dehydration catalysts or dehydration-functional constituents may comprise one or more metal oxides, for example those selected from the group consisting of aluminum oxide, silicon oxide, titanium oxide, zirconium oxide, magnesium oxide, calcium oxide, iron oxide, vanadium oxide, chromium oxide, nickel oxide, tungsten oxide, and strontium oxide. Such metal oxides may serve as a binder to provide a structured dehydration catalyst or dehydration-functional constituent, and these metal oxides may, more particularly, serve as a binder for the zeolitic and/or non-zeolitic molecular sieve, if used to form the dehydration catalyst or dehydration-functional constituent. In representative embodiments, the dehydration catalyst or dehydration-functional constituent may comprise (a) a single type of zeolitic molecular sieve or (b) a single type of non-zeolitic molecular sieve, with (a) or (b) optionally being in combination with (c) a single type of metal oxide. In this case, (a) or (b), and optionally (c), may be present in an amount, or optionally a combined amount, of greater than about 75 wt-% (e.g., from about 75 wt-% to about 99.9 wt-%) or greater than about 90 wt-% (e.g., from about 90 wt-% to about 99 wt-%), based on the weight of the dehydration catalyst or dehydration-functional constituent. For example, according to more particular embodiments, (a) or (b) alone may be present in these representative amounts.

The dehydration catalyst or a dehydration-functional constituent may further comprise one or more noble metal stabilizers or non-noble metal stabilizers (e.g., in elemental form, in the form of and oxide or other form) and/or one or more promoters selected from the group consisting of Mn, Mg, and/or Si (e.g., independently in elemental forms, oxide forms, or other forms), in respective amounts as described above. In general, in the case of a dehydration catalyst or dehydration-functional constituent of a bi-functional catalyst, the zeolitic and/or non-zeolitic molecular sieve(s), together with optionally one or more metal oxides as described above, stabilizers such as platinum or yttrium (e.g., in elemental form, in the form of an oxide or other form), and/or one or more promoters selected from the group consisting of manganese (Mn), magnesium (Mg), and silicon (Si) (e.g., independently in elemental forms, oxide forms, or other forms), may be present in a combined amount representing at least about 90%, at least about 95%, or at least about 99%, of the total weight of the dehydration catalyst or dehydration-functional constituent.

In the case of the dehydration catalyst or dehydration-functional constituent comprising a zeolite or a non-zeolitic molecular sieve, the zeolite or non-zeolitic molecular sieve may provide a solid support for components of this catalyst or functional constituent, such as one or more stabilizers and/or one or more promoters as described herein. For example, such stabilizer(s) and/or promoter(s) may be incorporated in the zeolite or non-zeolitic molecular sieve, acting as a solid support, according to various techniques for catalyst preparation, including sublimation, impregnation, or dry mixing. In the case of impregnation, an impregnation solution of soluble compounds of the one or more stabilizers and/or one or more promoters in a polar (aqueous) or non-polar (e.g., organic) solvent may be contacted with the solid support, preferably under an inert atmosphere. For example, this contacting may be carried out, preferably with stirring, in a surrounding atmosphere of nitrogen, argon, and/or helium, or otherwise in a non-inert atmosphere, such as air. The solvent may then be evaporated from the solid support, for example using heating, flowing gas, and/or vacuum conditions, leaving the dried, solid support comprising the zeolite or non-zeolitic molecular sieve and being impregnated with the stabilizer(s) and/or promoter(s). These components may be impregnated in the solid support, such as in the case of a plurality of metals (e.g., one or more stabilizers and one or more promoters, or otherwise two or more stabilizers or two or more promoters) being impregnated simultaneously by being dissolved in the same impregnation solution, or otherwise being impregnated separately using different impregnation solutions and contacting steps. In any event, the zeolite or non-zeolitic molecular sieve, acting as the solid support for impregnated stabilizer(s) and/or promoter(s) may be subjected to further preparation steps, such as washing with the solvent to remove excess metal(s) and impurities, further drying, calcination, etc. to provide the dehydration catalyst or dehydration-functional constituent. For incorporating any of a number of possible cations within a zeolite or non-zeolitic molecular sieve, as a component of a dehydration catalyst or dehydration-functional constituent, the technique of ion-exchange may be used. In this case, the support material is immersed in a solution of a cation, different from that already present in the exchange sites of the zeolite or non-zeolitic molecular sieve, and cations of the exchange sites are replaced with (exchanged by) cations of the solution, to equilibrium. The zeolite or non-zeolitic molecular sieve can then be washed to remove all ionic species that are not electrostatically bound to heteroatom exchange sites within the pores of the support material.

In yet further embodiments, regardless of the particular manner in which metals are incorporated/supported, as an alternative to supporting stabilizer(s) and/or promoter(s), or in addition to supporting stabilizer(s) and/or promoter(s), the zeolite or non-zeolitic molecular sieve may support one or more transition metals (e.g., one or more of Pt, Pd, Rh, Ir, and/or Au) in elemental form or in a compound form. Such one or more transition metals may be present in an amount, or combined amount, from about 0.05 wt-% to about 5 wt-%, such as from about 0.1 wt-% to about 3 wt-%, based on the weight of the transition metal(s), relative to the weight of the dehydration catalyst or dehydration-functional constituent comprising the zeolite or non-zeolitic molecular sieve. In the case of such transition metals being Pt and/or Pd, these may also be considered noble metal stabilizers. In still further embodiments, as an alternative to supporting stabilizer(s), promoter(s), and/or transition metal(s) or in addition to supporting stabilizer(s), promoter(s), and/or transition metal(s), the zeolite or non-zeolitic molecular sieve may support one or more surface-modifying agents (e.g., one or more of Si, Na, and/or Mg) in elemental form or in a compound form. Any surface-modifying agents, which by definition are disposed predominantly or completely on an external surface of the zeolite or non-zeolitic molecular sieve, may be present in an amount, or combined amount, from about 0.05 wt-% to about 5 wt-%, such as from about 0.1 wt-% to about 3 wt-%, based on the weight of the surface-modifying agent(s), relative to the weight of the dehydration catalyst or dehydration-functional constituent comprising the zeolite or non-zeolitic molecular sieve. In contrast to surface-modifying agents, any of the stabilizer(s), promoter(s), and/or transition metal(s) may be disposed uniformly throughout the zeolite or non-zeolitic molecular sieve used as a component of a dehydration catalyst or dehydration-functional constituent, or may be disposed according to any other profile (e.g., radial concentration profile), such as predominantly or completely on an external surface of the zeolite or non-zeolitic molecular sieve, or otherwise predominantly or completely within internal pores of such zeolite or non-zeolitic molecular sieve.

Those skilled in the art having knowledge of the present disclosure, including the general catalyst preparation procedures described above, will appreciate how such procedures can be adapted to obtain loadings of components (e.g., stabilizer(s), promoter(s), and/or transition metal(s)) with a desired profile, such as in the case of being concentrated near the external surface of, concentrated within internal pores of, or disposed uniformly throughout, a zeolite or non-zeolitic molecular sieve, with the desired profile likewise being applicable to the dehydration catalyst or dehydration-functional constituent as a whole. For example, an impregnation solution may be contacted with a powder form, or other finely divided form, of the zeolite or non-zeolitic molecular sieve to obtain a uniform distribution. Otherwise, in the case of using any one or more of the metal oxides described above as a binder for the zeolite or non-zeolitic molecular sieve, an impregnation solution may be contacted with larger, structured forms of the bound zeolite or non-zeolitic molecular sieve (e.g., having dimensions equivalent to, or on the same order as, the dehydration catalyst or dehydration-functional constituent as a whole) to obtain distributions of components preferentially near the external surface of the zeolite or non-zeolitic molecular sieve, or otherwise the dehydration catalyst or dehydration-functional constituent. The use of ion-exchange provides an effective method for incorporating stabilizers described herein (e.g., platinum) and other metals into a zeolite or non-zeolitic molecular sieve support material with a desirable distribution. An important characteristic of ion-exchange is the ability of this technique to deposit active metals as single (atomic) cations and at specific sites on the catalyst, namely those sites with a charge imbalance resulting from a heteroatom such as Al. The deposited metal is therefore atomically dispersed and present in a limited number of specific coordination environments.

In a representative dehydration catalyst or bi-functional catalyst, any metal(s) other than (a) Pt and/or Y (optionally together with any one or more other metals selected from Group 3 or Group 4 of the Periodic Table, and/or one or more lanthanides) and/or (b) metal(s) present in the zeolitic and/or non-zeolitic molecular sieve(s) and optionally one or more metal oxides as described above, may be present in minor amounts, may be substantially absent, or may be absent. For example, any such metal(s) other than (a) and/or (b) may be independently present in an amount of less than about 1 wt-%, less than about 0.1 wt-%, or even less than about 0.05 wt-%, based on the total catalyst weight. Alternatively, any two or more of such other metals may be present in a combined amount of less than about 2 wt-%, less than about 0.5 wt-%, or even less than about 0.1 wt-%, based on the total catalyst weight. According to particular embodiments, a dehydration catalyst or bi-functional catalyst may comprise metal(s) other than Pt and/or Y (optionally together with any one or more other metals selected from Group 3 or Group 4 of the Periodic Table, and/or one or more lanthanides), Mn, and/or Mg in the amounts described above; metals other than Pt and/or Y (optionally together with any one or more other metals selected from Group 3 or Group 4 of the Periodic Table, and/or one or more lanthanides), Mn, Mg, and/or Si in the amounts described above; metals other than Pt and/or Y (optionally together with any one or more other metals selected from Group 3 or Group 4 of the Periodic Table, and/or one or more lanthanides), Mn, Mg, Si, and/or P in the amounts described above; metals other than Pt and/or Y (optionally together with any one or more other metals selected from Group 3 or Group 4 of the Periodic Table, and/or one or more lanthanides), Mn, Mg, Si, P, Zn, Co, and/or Fe in the amounts described above; metals other than Pt and/or Y (optionally together with any one or more other metals selected from Group 3 or Group 4 of the Periodic Table, and/or one or more lanthanides), Mn, Mg, Si, P, Zn, Co, Fe, Al, Ti, Zr, Mg, and/or Ca in the amounts described above; or metals other than Pt and/or Y (optionally together with any one or more other metals selected from Group 3 or Group 4 of the Periodic Table, and/or one or more lanthanides), Mn, Mg, Si, P, Zn, Co, Fe, Al, Ti, Zr, Mg, Ca, V, Cr, Ni, W, and/or Sr in the amounts described above. For convenience, in these particular embodiments, Si and P will be considered “metals” in terms of their contributions to a dehydration catalyst or bi-functional catalyst. Other components of an alcohol synthesis catalyst (e.g., a methanol synthesis catalyst), a dehydration catalyst, or a bi-functional catalyst as described herein, such as binders (e.g., one or more metal oxides as described herein) and other additives, may be present in minor amounts, such as in an amount, or combined amount, of less than about 10 wt-% (e.g., from about 0.01 wt-% to about 10 wt-%), less than about 5 wt-% (e.g., from about 0.01 wt-% to about 10 wt-%), or less than about 1 wt-% (e.g., from about 0.01 wt-% to about 10 wt-%), based on the weight of the catalyst.

In the case of LPG synthesis catalyst systems comprising separate compositions of two different catalyst types, components of an alcohol synthesis catalyst (e.g., a methanol synthesis catalyst) as described herein may be substantially absent, or absent, from a dehydration catalyst. In the same manner, components of a dehydration catalyst as described herein may be substantially absent, or absent, from an alcohol synthesis catalyst (e.g., a methanol synthesis catalyst). For example, a representative dehydration catalyst may comprise (a) one or more alcohol synthesis-active metals (e.g., methanol synthesis-active metals(s)) described herein, (b) a solid support as described herein, (c) one or more stabilizers such as platinum and/or yttrium, and/or (d) one or more promoters of Mn, Mg, and/or Si, in an amount of (a), (b), (c), and/or (d), such as in a combined amount of (a), (b), (c), and (d), of less than about 5 wt-%, less than about 1 wt-%, or less than about 0.1 wt-%. This applies to dehydration catalysts generally, but this may also apply, more particularly, to dehydration catalysts of catalyst systems in which the alcohol synthesis catalyst (e.g., methanol synthesis catalyst) comprises, respectively, (a), (b), (c), and/or (d). For example, in the case of an alcohol synthesis catalyst (e.g., methanol synthesis catalyst) comprising (a) one or more alcohol synthesis-active metal(s) (e.g., methanol synthesis-active metals(s)), a dehydration catalyst of a catalyst system comprising that alcohol synthesis catalyst (e.g., methanol synthesis catalyst) may comprise such (a) one or more alcohol synthesis-active metal(s) (e.g., methanol synthesis-active metals(s)) in an amount, or combined amount, as described above (e.g., in an amount, or combined amount, of less than about 0.1 wt-%). Similarly, in the case of an alcohol synthesis catalyst (e.g., methanol synthesis catalyst) comprising (b), (c), and/or (d), a dehydration catalyst of a catalyst system comprising that alcohol synthesis catalyst (e.g., methanol synthesis catalyst) may comprise such respective (b), (c), and/or (d) in an amount, or combined amount, as described above. Alternatively, or in combination, a representative alcohol synthesis catalyst (e.g., methanol synthesis catalyst) may comprise (a) one or more zeolitic and/or non-zeolitic molecular sieve(s), (b) one or more metal oxides as described above, (c) one or more stabilizers such as platinum and/or yttrium, (d) one or more promoters of Mn, Mg, and/or Si, (e) one or more transition metals (e.g., Pt, Pd, Rh, Ir, and/or Au), and/or (f) one or more surface-modifying agents (e.g., Si, Na, and/or Mg) in an amount of (a), (b), (c), (d), (e), and/or (f) such as in a combined amount of (a), (b), (c), (d), (e), and/or (f) of less than about 5 wt-%, less than about 1 wt-%, or less than about 0.1 wt-%. This applies to alcohol synthesis catalysts (e.g., methanol synthesis catalysts) generally, but this may also apply, more particularly, to alcohol synthesis catalysts (e.g., methanol synthesis catalysts) of catalyst systems in which the dehydration catalyst comprises, respectively, (a), (b), (c), (d), (e), and/or (f). For example, in the case of a dehydration catalyst comprising (a) one or more zeolitic and/or non-zeolitic molecular sieve(s), an alcohol synthesis catalyst (e.g., methanol synthesis catalyst) of a catalyst system comprising that dehydration catalyst may comprise such (a) one or more zeolitic and/or non-zeolitic molecular sieve(s) in an amount, or combined amount, as described above (e.g., in an amount, or combined amount, of less than about 0.1 wt-%). Similarly, in the case of a dehydration catalyst comprising (b), (c), (d), (e), and/or (f), a methanol synthesis catalyst of a catalyst system comprising that dehydration catalyst may comprise such respective (b), (c), (d), (e), and/or (f) in an amount, or combined amount, as described above.

As an alternative to separate compositions of two different catalyst types, LPG synthesis may be performed using a single catalyst composition, namely a bi-functional catalyst comprising both an alcohol synthesis-functional constituent (e.g., methanol synthesis-functional constituent) and a dehydration-functional constituent. In terms of the compositions of these constituents, they may correspond in isolation to an alcohol synthesis catalyst (e.g., a methanol synthesis catalyst) and a dehydration catalyst, respectively, as described herein. When combined in a single catalyst composition, the functional constituents (i) and (ii) may be present in weight ratios as described herein. A representative bi-functional catalyst may therefore comprise (i) an alcohol synthesis-functional constituent (e.g., methanol synthesis-functional constituent) comprising one or more alcohol synthesis-active metals (e.g., methanol synthesis-active metals) as described above, and optionally a solid support as described herein, and (ii) a dehydration-functional constituent comprising a zeolite or non-zeolitic molecular sieve, and optionally a metal oxide, one or more transition metals, and/or one or more surface-modifying agents, as described herein. Either (i) or (ii) may further comprise a stabilizer, such as a noble metal stabilizer (e.g., platinum) or a non-noble metal stabilizer such as yttrium (e.g., in elemental form, in the form of an oxide such as yttria or other form) and/or one or more promoters selected from the group consisting of Mn, Mg, and/or Si (e.g., independently in elemental forms, oxide forms, or other forms).

It can be appreciated from the above description, including the weight ratios in which (i) and (ii) may be combined, that the one or more alcohol synthesis-active metals (e.g., methanol synthesis-active metals) may be present in a bi-functional catalyst as a whole, in an amount, or combined amounts, that is/are less than that/those amounts in which they are present in an alcohol synthesis catalyst (e.g., methanol synthesis catalyst), as described above. Likewise, the zeolite or non-zeolitic molecular sieve may be present in a bi-functional catalyst as a whole, in an amount that is less than that in which it is present in a dehydration catalyst, as described above. For example, a bi-functional catalyst as a whole may comprise the one or more alcohol synthesis-active metals (e.g., methanol synthesis-active metals) in lower amount, such as independently in an amount generally from about 0.2 wt-% to about 30 wt-%, typically from about 0.5 wt-% to about 15 wt-%, and often from about 0.8 wt-% to about 5 wt-%, based on the weight of the bi-functional catalyst. Likewise, a bi-functional catalyst as a whole may comprise (a) a single type of zeolitic molecular sieve or (b) a single type of non-zeolitic molecular sieve, with (a) or (b) optionally being in combination with (c) a single type of metal oxide. In this case, (a) or (b), and optionally (c), may be present in an amount, or optionally a combined amount, of greater than about 35 wt-% (e.g., from about 35 wt-% to about 95 wt-%), greater than about 50 wt-% (e.g., from about 50 wt-% to about 90 wt-%), or greater than about 75 wt-% (e.g., from about 75 wt-% to about 85 wt-%), based on the weight of the bi-functional catalyst. For example, according to more particular embodiments, (a) or (b) alone may be present in these representative amounts. Further in view of the above description, based on the weight of one or more stabilizers such as platinum and/or yttrium, and, relative to the weight of a bi-functional catalyst as a whole, the one or more stabilizers such as platinum and/or yttrium (e.g., in elemental form, in the form of an oxide or other form) may be present in an amount from about 0.01 wt-% to about 10 wt-%, such as from about 0.05 wt-% to about 6 wt-% or from about 0.1 wt-% to about 1 wt-%. Based on the weight of Mn, Mg, and/or Si, and, relative to the weight of the bi-functional catalyst as a whole, the promoter(s) (e.g., independently in elemental forms, oxide forms, or other forms) may be present in an amount from about 0.05 wt-% to about 12 wt-%, such as from about 0.1 wt-% to about 10 wt-% or from about 0.5 wt-% to about 8 wt-%.

Representative bi-functional catalysts may therefore comprise: (i) as an alcohol synthesis-functional constituent (e.g., methanol synthesis-functional constituent), one or more alcohol synthesis-active metals (e.g., methanol synthesis-active metal(s)), or any forms of such metals (e.g., elemental and/or oxide forms), as described herein, and optionally any solid support as described herein, and (ii) as a dehydration-functional constituent, one or more zeolitic and/or non-zeolitic molecular sieve(s) as described herein, and optionally one or more metal oxides, one or more transition metals, and/or one or more surface-modifying agents, as described herein. Such bi-functional catalyst may further comprise, for example as component(s) of either the alcohol synthesis-functional constituent (e.g., methanol synthesis-functional constituent) and/or the dehydration-functional constituent, a stabilizer such as platinum and/or yttrium (e.g., in elemental form, in the form of an oxide or other form), and/or one or more promoters of Mn, Mg, and/or Si (e.g., independently in elemental forms, oxide forms, or other forms). The one or more alcohol synthesis-active metals (e.g., methanol synthesis-active metal(s)), or any forms of such metals (e.g., elemental and/or oxide forms), one or more zeolitic and/or non-zeolitic molecular sieve(s), together with any optional solid support, optional metal oxide(s), optional transition metal(s), optional surface-modifying agent(s), stabilizers, such as a noble metal stabilizer (e.g., platinum) or a non-noble metal stabilizer such as yttrium (e.g., in elemental form, in the form of an oxide such as yttria or other form), and/or one or more promoters of Mn, Mg, and/or Si (e.g., independently in elemental forms, oxide forms, or other forms), may constitute all or substantially all of the bi-functional catalyst, for example these components may be present in a combined amount representing at least about 90%, at least about 95%, or at least about 99%, of the total weight of the bi-functional catalyst.

Conditions used in processes for producing an LPG product, and more particularly conditions under which LPG synthesis catalyst systems, as described herein, are maintained (e.g., in one or more LPG synthesis reactors), are suitable for the conversion of H₂ and CO in a synthesis gas to propane and/or butane of the LPG product. In representative embodiments, such LPG synthesis reaction conditions, suitable for use in at least one LPG synthesis reactor or, more particularly, one or more catalyst beds contained in such reactor(s), can include an LPG synthesis reaction temperature, corresponding to an initial operation temperature in step (b) as described above, or otherwise a temperature used at another time, such as a subsequent operation temperature as described above. Such temperature may be used to provide a given level of performance (e.g., selectivity to and/or yield of LPG hydrocarbons) during some operational period of the process (e.g., following at least some deactivation due to use of the LPG synthesis catalyst system, which has occurred prior to this operational period). Representative LPG synthesis reaction temperatures may range from about 204° C. (400° F.) to about 454° C. (850° F.), or from about 316° C. (600° F.) to about 399° C. (750° F.). These temperatures may be understood as referring to, in various embodiments, inlet temperature, peak temperature, or WABT, as described above. An LPG synthesis reaction pressure, suitable for use in at least one LPG synthesis reactor, can include a gauge pressure from about 690 kPa (100 psig) to about 6.9 MPa (1000 psig), such as from about 1.38 MPa (200 psig) to about 2.76 MPa (400 psig) or from about 3.4 MPa (500 psig) to about 5.2 MPa (750 psig). Such pressures may be used in any step of the process, for example in activation step (a), operating step (b), or in subsequent operation.

The LPG synthesis catalyst systems and LPG synthesis reaction conditions described herein are generally suitable for achieving a conversion of H₂ and/or CO (H₂ conversion or CO conversion) of at least about 20% (e.g., from about 20% to about 99% or from about 20% to about 95%), at least about 30% (e.g., from about 30% to about 99% or from about 30% to about 95%), or at least about 50% (e.g., from about 50% to about 95% or from about 75% to about 95%). As is understood in the art, the conversion of H₂ or CO in a synthesis gas can be calculated on the basis of:

100*(H_{2feed}−H_{2prod})/H_{2feed} or 100*(CO_{feed}−CO_{prod})/CO_{feed}

wherein H_{2feed} or CO_{feed} is the total amount (e.g., total weight or total moles) of H₂ or CO, respectively, in the synthesis gas provided to one or more LPG synthesis reactors containing an LPG synthesis catalyst system as described herein, and H_{2prod} or CO_{prod} is the total amount of H₂ or CO, respectively, in the effluent from the reactor(s), which may, but does not necessarily, correspond to the total amount of H₂ or CO in the LPG product. In the case of continuous processes, these total amounts may be more conveniently expressed in terms of flow rates, or total amounts per unit time (e.g., total weight/hr or total moles/hr). These H₂ or CO conversion levels may be based on “per-pass” conversion, achieved in a single pass through one or more LPG synthesis reactors, or otherwise based on overall conversion, achieved by returning a recycle portion of the effluent, containing unconverted H₂ and/or CO (and possibly enriched in these unconverted reactants, relative to the effluent and/or the LPG product), back to the LPG synthesis reactor(s). Whether these LPG synthesis conversion levels are based on H₂ conversion or CO conversion may depend on which reactant is stoichiometrically limited in the synthesis gas being fed or introduced to the LPG synthesis reactor(s), considering the LPG synthesis reaction chemistry. Preferably, these LPG synthesis conversion levels are based on CO conversion, or conversion of CO in the synthesis gas. These LPG synthesis conversion levels may be attained in any step of the process, for example in activation step (a), operating step (b), or in subsequent operation. In some cases, these LPG synthesis conversion levels are attained in any combination of these steps, or in all of these steps, but not in a pre-activation step as described above. In some cases, these LPG synthesis conversion levels are attained in operating step (b) and in subsequent operation, but not activation step (a) or in a pre-activation step as described above, particularly in cases in which the activating gas is other than a synthesis gas comprising H₂ and CO.

Another important performance parameter with respect to processes as described herein for producing an LPG product is carbon selectivity to LPG hydrocarbons, which refers to percentage of carbon (e.g., present in CO and CO₂) that is fed or introduced to the LPG synthesis reactor(s) and that manifests in LPG hydrocarbons, namely propane and/or butane (including both of the butane isomers, iso- and normal-butane) in the effluent from the reactor(s), which may, but does not necessarily, correspond to this percentage that manifests in LPG hydrocarbons in the LPG product. In representative embodiments, carbon selectivity to LPG hydrocarbons is at least about 20% (e.g., from about 20% to about 90% or from about 20% to about 75%), at least about 30% (e.g., from about 30% to about 90% or from about 30% to about 75%), at least about 40% (e.g., from about 40% to about 90% or from about 40% to about 75%), or even at least about 50% (e.g., from about 50% to about 90% or from about 50% to about 75%). The carbon selectivity to propane may be at least about 10% (e.g., from about 10% to about 60% or from about 10% to about 50%), at least about 15% (e.g., from about 15% to about 60% or from about 15% to about 50%), or at least about 20% (e.g., from about 20% to about 60% or from about 20% to about 50%). The carbon selectivity to butane (both iso- and normal-butane) may be at least about 5% (e.g., from about 5% to about 45% or from about 5% to about 35%), at least about 10% (e.g., from about 10% to about 45% or from about 10% to about 35%), or at least about 15% (e.g., from about 15% to about 45% or from about 15% to about 35%). These carbon selectivity levels may be attained in any step of the process, for example in activation step (a), operating step (b), or in subsequent operation. In some cases, these carbon selectivity levels are attained in any combination of these steps, or in all of these steps, but not in a pre-activation step as described above. In some cases, these carbon selectivity levels are attained in operating step (b) and in subsequent operation, but not in activation step (a) or in a pre-activation step as described above, particularly in cases in which the activating gas is other than a synthesis gas comprising H₂ and CO.

A per-pass (or single pass) yield of LPG hydrocarbons provides a further, important measure of performance of representative processes as described herein. This per-pass yield refers to the product of the per-pass CO conversion and the carbon selectivity to LPG hydrocarbons. In representative processes, the per-pass yield of LPG hydrocarbons (or LPG hydrocarbon yield) is at least about 15% (e.g., from about 15% to about 85% or from about 15% to about 70%), at least about 25% (e.g., from about 25% to about 85% or from about 25% to about 70%), at least about 35% (e.g., from about 35% to about 85% or from about 35% to about 70%), or even at least about 45% (e.g., from about 45% to about 85% or from about 45% to about 70%). In some preferred embodiments, the per-pass yield of LPG hydrocarbons in the LPG synthesis stage is at least about 50%. These per-pass yields of LPG hydrocarbons may be attained in any step of the process, for example in activation step (a), operating step (b), or in subsequent operation. In some cases, these per-pass yields of LPG hydrocarbons are attained in any combination of these steps, or in all of these steps, but not in a pre-activation step as described above. In some cases, these per-pass yields of LPG hydrocarbons are attained in operating step (b) and in subsequent operation, but not in activation step (a) or in a pre-activation step as described above, particularly in cases in which the activating gas is other than a synthesis gas comprising H₂ and CO.

A desired H₂ conversion and/or CO conversion in the LPG synthesis reactor(s), as well as other desired performance parameters, may be achieved, for example in any of activation step (a), operating step (b), and/or subsequent operation, by adjusting the LPG synthesis reaction conditions described above (e.g., LPG synthesis reaction temperature and/or LPG synthesis reaction pressure), and/or adjusting the weight hourly space velocity (WHSV). As is understood in the art, the WHSV is the weight flow of the synthesis gas divided by the total weight of catalyst in the LPG synthesis catalyst system (e.g., present in a fixed bed or other reactor bed configuration in the LPG synthesis reactor(s)) and represents the equivalent catalyst bed weights of the synthesis gas processed per hour. The WHSV is related to the inverse of the reactor residence time. The LPG synthesis reaction conditions may include a weight hourly space velocity (WHSV) generally less than about 10 hr⁻¹ (e.g., from about 0.01 hr⁻¹ to about 10 hr⁻¹), typically less than about 5 hr⁻¹ (e.g., from about 0.05 hr⁻¹ to about 5 hr⁻¹), and often less than about 1.5 hr⁻¹ (e.g., from about 0.1 hr⁻¹ to about 1.5 hr⁻¹), as defined above. These values of WHSV may be used in operating step (b) and/or in subsequent operation. These values of WHSV may likewise be used in a pre-activation step and/or in an activation step (a), with such values, in the case of using an activating gas, being defined in a manner analogous to the definition above, i.e., being based on the weight flow of the activating gas divided by the total weight of catalyst in the LPG synthesis catalyst system. Regardless of whether WHSV is based on the weight flow of the synthesis gas or the activating gas, as an alternative to being based on the entire weight of the catalyst in the LPG synthesis catalyst system, the WHSV may be based on the combined weight of a methanol synthesis catalyst and a dehydration catalyst, or otherwise based on the weight of a bi-functional catalyst, as described herein. The conversion level (e.g., CO conversion) may be increased, for example, by increasing pressure and decreasing WHSV, having the effects, respectively, of increasing reactant concentrations and reactor residence times.

In some embodiments, conditions of one or both of pressure and WHSV, in operating step (b), are the same as, or comparable to, those in activation step (a). By being “comparable to,” the absolute pressure in operating step (b) may be +/−20% of, such as +/−10% of, the absolute pressure in activation step (a) and/or the WHSV in operating step (b) may be +/−20% of, such as +/−10% of, the WHSV in activation step (a). Alternatively, or in combination, conditions of one or both of pressure and WHSV, in operating step (b), are the same as, or comparable to, those in (i) a pre-activation step as described herein, and/or (ii) subsequent operation, as described herein. With respect to such condition(s) of operating step (b) being the same or comparable, this may, according to some embodiments, be in reference to the corresponding condition(s) during at least a portion of a pre-activation step, an activation step (a), and/or subsequent operation. This may, according to other embodiments, be in reference to the corresponding condition(s) over the entire duration of a pre-activation step, an activation step (a), and/or subsequent operation.

The LPG product may be an LPG synthesis effluent, i.e., the effluent from one or more LPG synthesis reactors (e.g., the LPG product may be obtained without further processing of the LPG synthesis effluent) or otherwise the LPG product may be separated from the LPG synthesis effluent, for example as a fraction of the LPG synthesis effluent that is enriched in propane and/or butane and that is separated using techniques known in the art (e.g., fractionation). In either case, the LPG synthesis effluent may be obtained directly from an LPG synthesis reactor that contains an LPG synthesis catalyst system, or at least one catalyst of such system (e.g., an alcohol synthesis catalyst, such as a methanol synthesis catalyst, or a dehydration catalyst), as described herein. In preferred embodiments, processes described herein comprise a step of separating the LPG product from the LPG synthesis effluent. In addition to this LPG product, processes may further comprise separating one or more other fractions from the LPG synthesis effluent, such as fractions that are depleted in LPG hydrocarbons, relative to the LPG product. For example, such other fraction(s) may include an H₂/CO-enriched fraction, i.e., a fraction that is enriched in H₂ and CO, relative to the LPG synthesis effluent and the LPG product. Such other fraction(s) may, alternatively or in combination, include a water-enriched fraction, i.e., a fraction that is enriched in water, relative to the LPG synthesis effluent and the LPG product. Both such H₂/CO-enriched fraction and water-enriched fraction represent fractions that, following their separation from the LPG synthesis effluent, may advantageously be recycled in the process. The H₂/CO₂-enriched fraction and water-enriched fractions may, respectively, represent gaseous (vapor) and liquid fractions separated from the LPG synthesis effluent, e.g., as respective, lower-boiling (more volatile) and higher-boiling (less volatile) fractions, relative to the LPG product. One or more of these separations may be performed in any step of the process, for example in activation step (a), operating step (b), or in subsequent operation. In some cases, one or more of these separations may be performed in any combination of these steps, or in all of these steps, but not in a pre-activation step as described above. In some cases, one or more of these separations may be performed in operating step (b) and in subsequent operation, but not in activation step (a) or in a pre-activation step as described above, particularly in cases in which the activating gas is other than a synthesis gas comprising H₂ and CO.

According to specific embodiments, the LPG product (e.g., following a step of separating the LPG product from the LPG synthesis effluent) may comprise propane and butane in a combined amount of at least about 60 mol-% (e.g., from about 60 mol-% to about 100 mol-%), at least about 80 mol-% (e.g., from about 80 mol-% to about 100 mol-%), or at least about 90 mol-% (e.g., from about 90 mol-% to about 99 mol-%). Together with such combined amounts, or alternatively, the LPG product may comprise propane and/or butane independently in individual amounts of at least about 25 mol-% (e.g., from about 25 mol-% to about 85 mol-%), at least about 40 mol-% (e.g., from about 40 mol-% to about 80 mol-%), or at least about 50 mol-% (e.g., from about 50 mol-% to about 75 mol-%). The balance of the LPG product may comprise all, or substantially all, pentane or a combination of ethane and pentane. According to other specific embodiments, at least about 40% (e.g., from about 40% to about 95%), at least about 55% (e.g., from about 55% to about 95%), or at least about 70% (e.g., from about 70% to about 95%) of the carbon content of the synthesis gas (e.g., the carbon content of CO and/or CO₂ present in this mixture) forms propane and/or butane of the LPG product. These percentages are equivalently expressed in terms of wt-% or mol-%.

### EXAMPLES

The following examples are set forth as representative of the present invention. These examples are not to be construed as limiting the scope of the invention as other equivalent embodiments will be apparent in view of the present disclosure and appended claims.

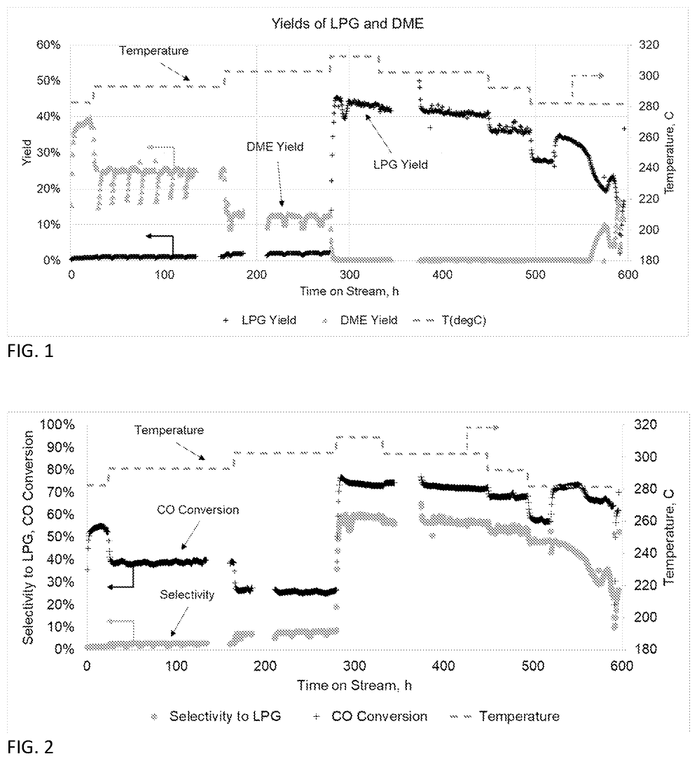

An LPG synthesis catalyst system with 6 grams total of methanol synthesis catalyst (Cu/ZnO/Al₂O₃) and zeolite beta was tested for conversion of a synthesis gas (13 mol-% CO, 67 mol-% H₂, 17 mol-% CO₂, and 3 mol-% N₂) at 2.1 MPa (300 psig) gauge pressure. A normal flow rate of this synthesis gas was maintained at 70 ml/min. The results shown in FIG. 1 illustrate that essentially only DME was produced initially, up to about 280 hours on stream (HOS) at pre-activation temperatures as high as 300° C. The apparent, periodic “dips” in DME yield resulted from the particular sampling protocol, whereby methanol was periodically observed in the product condensate. The LPG synthesis catalyst system thereafter became activated at an activation temperature of 310° C., which initiated the desired methanol to hydrocarbons chemistry (presumably due to formation of the hydrocarbon pool). Following this activation, the bed temperature of the LPG synthesis catalyst system could be reduced to an initial operation temperature of 290° C., or even 280° C., while maintaining LPG hydrocarbon yields that were not observed at these temperatures, prior to activation. As illustrated in FIG. 2, following activation, the CO conversion and LPG hydrocarbon selectivity were stable. Performance data were not evaluated for a short operating period around 350-375 HOS, as also seen in FIGS. 1 and 2.

Importantly, it is estimated that the slope of the deactivation curve (represented by the % CO conversion loss per day), with a 290° C. initial operation temperature, is less than 10% of that obtained in the case of performing LPG synthesis at 310° C. This would have been the expected initial operation temperature, given the behavior of the LPG synthesis catalyst system and in the absence of discovering that the temperature could be reduced, following activation, without reverting to the same pre-activation performance levels. Whereas LPG hydrocarbon yield, following activation, is reduced to some extent at lower temperature in view of reaction kinetics, the thermodynamics of methanol synthesis allow for offsetting of this performance deficit, by increasing pressure at these lower temperatures. In any event, by performing the process at an initial activation temperature of 290° C., as opposed to 310° C., a significant improvement in process economics can be realized, at least in terms of reduced catalyst replacement costs that are associated with a longer catalyst operating life.

As further illustrated in FIG. 1, following activation that was marked by an abrupt increase in LPG hydrocarbon yield to above 40% at an activation temperature of 310° C., significant LPG production was maintained even after decreasing temperature to 300° C., then to 290° C., and finally to 280° C., any of which lower, subsequent temperatures could be considered an “initial operation temperature,” according to embodiments described herein. At 280° C., the production of LPG hydrocarbons gradually decreased over a period of about 80 hours, with a measurable increase in DME production towards the end of this period. However, the LPG synthesis catalyst system still proved responsive upon returning the temperature to 310° C., with a resumption of LPG production and suppression of DME production, shortly before reaching 600 HOS.

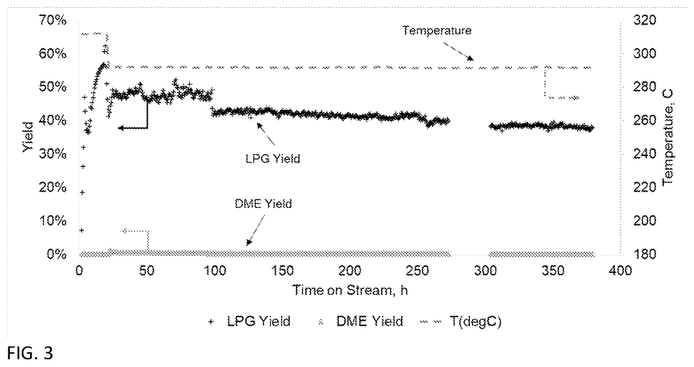

As illustrated in FIG. 3, operation with favorable performance in terms of LPG hydrocarbon yield (with little or no DME yield) at temperatures below 300° C., was possible for an extended period of time, following activation of the LPG synthesis catalyst system at higher temperatures (310° C. or above). This additional experiment with the LPG synthesis catalyst system, synthesis gas composition, pressure, and flow rate as described above, was run continuously for almost 400 HOS (with the gap in the data around 275-305 HOS being due to loss of analytical capabilities), utilizing a different temperature versus time profile. Furthermore, the rate of catalyst deactivation was directionally higher at higher temperatures. Between 100 HOS and 380 HOS, at a temperature of 290° C., the deactivation rate was estimated to be 0.47% CO conversion loss per day, whereas at 310° C. and 350° C., deactivation was estimated to be 0.60% and 1.3%, respectively, CO conversion loss per day with the same catalyst system and operating conditions (aside from temperature).

Overall, aspects of the invention relate to the surprising discovery the LPG synthesis catalyst systems can be activated for LPG hydrocarbon product, prior to reducing temperature to an initial operation temperature at which favorable performance can be attained, in addition to advantageous results in terms of catalyst stability. Those skilled in the art, having knowledge of the present disclosure, will recognize that various changes can be made to LPG production processes, including operating conditions and LPG synthesis catalyst systems used, in attaining these and other advantages, without departing from the scope of the present disclosure. As such, it should be understood that the features of the disclosure are susceptible to modifications and/or substitutions without departing from the scope of this disclosure. The specific embodiments illustrated and described herein are for illustrative purposes only, and not limiting of the invention as set forth in the appended claims.

1. A process for producing an LPG product comprising propane and/or butane, the process comprising:
(a) contacting an activating gas with an LPG synthesis catalyst system at an activation temperature or above, and thereafter
(b) contacting a synthesis gas comprising H₂ and CO with the LPG synthesis catalyst system at an initial operation temperature that is less than the activation temperature,
wherein the LPG synthesis catalyst system comprises:
(i) an alcohol synthesis catalyst, and
(ii) a dehydration catalyst.

2. The process of claim 1, wherein the alcohol synthesis catalyst is a methanol synthesis catalyst.

3. A process for producing an LPG product comprising propane and/or butane, the process comprising:
(a) contacting an activating gas with an LPG synthesis catalyst system at an activation temperature or above, and thereafter
(b) contacting a synthesis gas comprising H₂ and CO with the LPG synthesis catalyst system at an initial operation temperature that is less than the activation temperature,
wherein the LPG synthesis catalyst system comprises a bi-functional catalyst, having as constituents:
(i) an alcohol synthesis-functional constituent, and
(ii) a dehydration-functional constituent.

4. The process of claim 3, wherein the alcohol synthesis-functional constituent is a methanol synthesis-functional constituent.

5. The process of claim 1 or claim 3, wherein the activating gas comprises or generates at least one of H₂, CO, and CO₂.

6-7. (canceled)

8. The process of claim 1 or claim 3, wherein the activating gas comprises a hydrocarbon or an oxygenated hydrocarbon.

9-11. (canceled)

12. The process of claim 1 or claim 3, wherein the activation temperature is at least 295° C.

13-14. (canceled)

15. The process of claim 1 or claim 3, wherein step (a) is carried out for an activation period sufficient to obtain an activation LPG hydrocarbon yield of at least about 20%.

16. (canceled)

17. The process of claim 1 or claim 3, wherein step (a) is carried out for an activation period of at least about 1 hour.

18. The process of claim 1 or claim 3, wherein, in a reference process consisting of step (b) in the absence of step (a), a reference initial LPG hydrocarbon yield is less than an initial operation LPG hydrocarbon yield in step (b) of the process.

19. (canceled)

20. A process for producing an LPG product comprising propane and/or butane, the process comprising contacting a synthesis gas comprising H₂ and CO with an LPG synthesis catalyst system at an LPG synthesis catalyst system temperature that is a pre-activation temperature, sufficient to obtain a pre-activation methanol and/or dimethyl ether (DME) yield of at least about 20%, the process further comprising:
(a) increasing the LPG synthesis catalyst system temperature to an activation temperature or above, sufficient to provide an activation LPG hydrocarbon yield of at least about 30%; and
(b) decreasing the LPG synthesis catalyst system temperature to an initial operation temperature, sufficient to maintain an initial operation LPG hydrocarbon yield of at least about 30%.

21. (canceled)

22. The process of claim 20, wherein, in a reference process consisting of said contacting at the pre-activation temperature and step (a), but in the absence of step (b), a reference deactivation rate of the LPG synthesis catalyst system is greater than an operating deactivation rate of the process.

23. (canceled)

24. The process of claim 20, wherein the LPG synthesis catalyst system comprises:
(i) a methanol synthesis catalyst, and
(ii) a dehydration catalyst.

25. The process of claim 20, wherein the LPG synthesis catalyst system comprises a bi-functional catalyst, having as constituents:
(i) an alcohol synthesis-functional constituent, and
(ii) a dehydration-functional constituent.

26-34. (canceled)
